(19) United States
(12) Patent Application Publication
(10) Pub. No.: US 2024/0191290 A1
(43) Pub. Date: Jun. 13, 2024

(54) METHODS FOR DETECTION AND REDUCTION OF SAMPLE PREPARATION-INDUCED METHYLATION ARTIFACTS

(71) Applicant: GUARDANT HEALTH, INC., Palo Alto, CA (US)

(72) Inventors: Andrew KENNEDY, San Diego, CA (US); Annie M. DEMAREE, Palo Alto, CA (US)

(21) Appl. No.: 18/357,022

(22) Filed: Jul. 21, 2023

Related U.S. Application Data
(60) Provisional application No. 63/513,250, filed on Jul. 12, 2023.
(60) Provisional application No. 63/391,213, filed on Jul. 21, 2022.

(51) Int. Cl. C12Q 1/6869; C12Q 1/6886

(52) U.S. Cl. CPC C12Q 1/6869; C12Q 1/6886; C12Q 2600/154; C12Q 2600/156

(57) ABSTRACT

The disclosure relates to improved sequencing reactions. Specifically, the disclosure provides methods which allow for the identification of regions of a DNA molecule that were synthesized during an end repair and/or A-tailing reaction. Sequence data deriving from such synthesized regions may not be representative of the corresponding region in the original DNA molecules, for example it may contain artifactual methylation statuses. Accordingly, the identification of these synthesized regions allows for such potentially artifactual data to be identified and filtered accordingly.

### CROSS-REFERENCE TO RELATED APPLICATIONS

This application claims the benefit of priority of U.S. Provisional Patent Application No. 63/391,213, filed Jul. 21, 2022, and U.S. Provisional Patent Application No. 63/513,250 filed Jul. 12, 2023, each of which is incorporated by reference herein in its entirety for all purposes.

### FIELD OF THE INVENTION

The disclosure relates to methods for identifying regions of sequenced DNA that are synthesized during end-repair processes, which are commonly present in sequencing workflows. Such methods are important for accurately detecting the methylation status and variants present in DNA, which, in turn, can be important for inferring information about the cells and subject from which the DNA sample is derived.

### BACKGROUND

DNA end repair is a common step in sequencing workflows to prepare the DNA for adapter ligation. Sample DNA molecules typically contains a mixture of blunt ends, 3′end overhangs and 5′end overhangs. The end repair step normalizes these ends by removing 3′overhangs and filling in 5′overhangs to generate blunt ended DNA, which can then be subjected to blunt-ended adapter ligation or A-tailed before sticky-end ligation. Such end repair steps are commonly used in sequencing workflows, such as those used for mutation analysis and/or those used to determine the methylation status of nucleotides at an individual base resolution.

For example, single-nucleotide resolving assays to detect nucleoside methylation often require a conversion of the modified nucleosides or corresponding unmodified nucleosides to change their base-pairing specificity. The conversion is then detected by sequencing. Examples of such methods include bisulfite and oxidative bisulfite and Tet-assisted bisulfite conversion, EM-seq, TAPS and TAPS R conversion, ACE-seq and SEM-seq. See, e.g., Moss et al., Nat Commun. 2018; 9: 5068; Booth et al., Science 2012; 336: 934-937; Yu et al., Cell 2012; 149: 1368-80; Liu et al., Nature Biotechnology 2019; 37:424-429; Schutsky, E. K. et al.; Vaisvila et al. Genome Research 2021 31(7): 1280-1289; and Vaisvila, et al. 2023. Discovery of novel DNA cytosine deaminase activities enables a nondestructive single-enzyme methylation sequencing method for base resolution high-coverage methylome mapping of cell-free and ultra-low input DNA. bioRxiv doi: 10.1101/2023.06.29.547047 (doi.org/10.1101/2023.06.29.547047).

Bisulfite-based, EM-seq and SEM-seq methylation assays convert unmethylated cytosine to uracil, which is PCR-amplified and read as thymine in sequencing reactions. In other methods, the epigenetic conversion is opposite, i.e. the modified nucleosides, rather than the unmodified nucleosides, are converted. For example, in the TAPS method, methylated cytosines (5mCs and 5hmCs) are converted to dihydrouridine (DHU), PCR-amplified and read as thymine in sequencing reactions. Single molecule sequencing technologies, such a nanopore-based sequencing and single molecule real time sequencing (SMRT) sequencing can alternatively be used to detect the modification status of nucleosides. These single molecule technologies can directly detect specific modification statuses of nucleosides and thus do not rely on the conversion of the modified nucleosides or corresponding unmodified nucleosides to change their base-pairing specificity.

During the end repair step, repair and synthesis of regions of the DNA molecule can occur which mean that the modification status of the nucleosides in these regions of the end-repaired DNA may not accurately reflect the modification status of the corresponding modification status in the original DNA fragment. Similarly, the templated nature of end repair can mean that it eradicates any mismatches between complementary strands (e.g. as a result of a single-stranded mutation). This can falsely lead to the conclusion that a detected sequence has double-stranded support (i.e. there is sequence data from both strands of the original DNA molecule that supports that sequence). Consequently, using end repair can lead to artifactual information which is not representative of the original DNA fragment, which, in turn, could lead to incorrect inferences in relation to the DNA sample and the subject from which the DNA sample was obtained.

Given the issues surrounding the artifactual information in these assays, there is a need for improved methods which allow for the identification of sequencing data which is known to accurately reflect the original DNA fragment.

### SUMMARY

The present disclosure provides embodiments including methods that allow sequence data from an original DNA molecule to be distinguished from sequence data from a region of a DNA molecule that has been synthesized during an end repair reaction (“synthesized regions”). This method can therefore be used to avoid using sequence data which derives from these synthesized regions to determine the properties of the original DNA molecule. For example, if the method was being used for determining the methylation statuses of cytosine in the original DNA molecule, the observed methylation status in the synthesized regions may not accurately reflect the methylation status of the corresponding nucleoside in the original DNA molecule. The method can therefore be used to determine the modification status of nucleosides using data which is known to derive from the original DNA molecule (i.e. not the synthesized regions) and thus accurately represent the methylation status of the nucleosides in the original DNA molecule. Similarly, the identification of such synthesized regions can be used to interpret sequencing data related to variant detection. For example, because the synthesized region is templated from the complementary strand, it will erase any base mismatches that were present in this region in the original DNA molecule. As such, any variants detected within the synthesized region cannot be confidently classified as having double-stranded support (i.e. sequence data from both strands of the original DNA molecule that supports that variant).

The disclosed methods achieve this by using at least one type of dNTP which comprises a modified base (e.g. a methylated deoxycytidine triphosphate, such as deoxycytidine triphosphate comprising 5-methylcytosine (5mC) and/or 5-hydroxymethyl-cytosine (5hmC)) in the end repair reaction. During end repair reactions, the methylated deoxycytidine triphosphate will be incorporated into the synthesized regions regardless of the sequence context. This will result in methylated cytosines in non-CpG positions, which are very rare in nature. These methylated non-CpG cytosines can therefore be used as labels for identifying synthesized regions in the end repaired DNA molecule. Similarly, other types of dNTP which comprise a modified base uncommon or absent in nature can be used. The identification of such modified bases can be performed using modification-sensitive sequencing, and regions comprising these modifications can be interpreted as defining regions which were synthesized in the end repair reaction.

Subsequent sequence analysis of the DNA sample can then be focused on the regions of the original DNA molecule that are known not to be synthesized during the end repair reaction, and thus accurately reflect the properties (e.g. modification status) of the original DNA molecule. This method therefore provides sequencing data with improved accuracy because it allows the subsequent sequence analysis to be focused on regions which do not contain the artifactual information (e.g. methylation statuses) associated with the regions of the DNA which are synthesized during the end repair reaction. The method can also be used to quantify the level of DNA damage in the original DNA molecule through the quantitative analysis of the synthesized region.

In one aspect, the disclosure provides a method comprising: (a) subjecting a DNA sample to end repair to generate end-repaired DNA, wherein the end repair is performed with deoxynucleotide triphosphates (dNTPs), wherein at least one type of dNTP comprises a modified base; (b) performing a ligation reaction to ligate adapters to the end-repaired DNA to generate adapted DNA, wherein the ligation reaction also seals nicks present in the end-repaired DNA; (c) subjecting the adapted DNA to modification-sensitive sequencing to obtain sequencing data derived from the DNA sample, wherein the modification-sensitive sequencing is capable of identifying the base modification in the at least one type of dNTP; and (d) analyzing the sequence data obtained in step (c) to identify one or more regions of the end-repaired DNA that were synthesized during the end repair by the presence of the base modification in the at least one type of dNTP used in step (a).

In some embodiments, the end repair is performed with a DNA polymerase which does not have 5′-3′ exonuclease activity and/or is not a strand displacing DNA polymerase. In some embodiments, the DNA polymerase is T4 DNA polymerase, T7 DNA polymerase or Klenow fragment. In some embodiments, the end repair is performed with a DNA polymerase which has 5′-3′ exonuclease activity and/or is a strand displacing DNA polymerase. In some embodiments, the at least one type of dNTP which comprises a modified base includes a dNTP comprising 4-methylcytosine (4mC), a dNTP comprising 5-methylcytosine (5mC), a dNTP comprising 5-hydroxymethyl-cytosine (5hmC), a dNTP comprising N6-methyladenosine (6 mA), a dNTP comprising bromodeoxyuridine (BrdU) and/or a dNTP comprising 8-oxoguanine (8oxoG). In some embodiments, the method further comprises between steps (a) and (b) performing an A-tailing reaction.

In some embodiments, the end-repair and the A-tailing reaction is performed in a single tube. In some embodiments, the A-tailing is performed using a DNA polymerase that does not possess 5′-3′ exonuclease activity and/or is not a strand displacing DNA polymerase, optionally the DNA polymerase is HemoKlen Taq. In some embodiments, the A-tailing is performed using a Taq DNA polymerase, Tfl DNA polymerase, Bst DNA Polymerase, Large Fragment or Tth DNA polymerase.

In some embodiments, the end-repair and the A-tailing reaction are performed as separate reactions, wherein a reaction clean-up step is performed after the end-repair and before the A-tailing reaction. In some embodiments, the A-tailing is performed using a DNA polymerase that does not possess 3′-5′ exonuclease activity, such as Klenow Fragment lacking 3′-5′ exonuclease activity. In some embodiments, the A-tailing is performed using a DNA polymerase that possesses 5′-3′ exonuclease activity and/or is a strand displacing DNA polymerase. In some embodiments, the ligation reaction is a blunt-end ligation reaction. In some embodiments, the ligation reaction is a sticky-end ligation reaction.

In some embodiments, the modification-sensitive sequencing comprises a conversion procedure that changes the base pairing specificity of the base or does not change the base pairing specificity of the base, depending on the modification status of the base. In some embodiments, the modified base is a methylated cytosine and wherein the conversion procedure converts methylated cytosines, for example the conversion procedure is Tet-assisted conversion with a substituted borane reducing agent, optionally wherein the substituted borane reducing agent is 2-picoline borane, borane pyridine, tert-butylamine borane, or ammonia borane. In some embodiments, the modified base is a methylated cytosine and the conversion procedure converts unmethylated cytosines, for example the conversion procedure is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq) or single-enzyme 5-methylctyosine sequencing (SEM-seq). In some embodiments, the modification-sensitive sequencing comprises nanopore-based sequencing or single-molecule real time (SMRT) sequencing. In some embodiments, the modification-sensitive sequencing comprises nanopore-based sequencing and the at least one type of dNTP which comprises a modified base includes a dNTP comprising 4mC (4-methyldeoxycytidine), a dNTP comprising 5mC (5-methyldeoxycytidine), a dNTP comprising 5hmC (5-hydroxymethyldeoxycytidine), a dNTP comprising 6 mA (6-methyldeoxyadenosine), a dNTP comprising BrdU (5-bromodeoxyuridine), dUTP, a dNTP comprising FldU (5-fluorodeoxyuridine), a dNTP comprising IdU (5-iododeoxyuridine), and/or a dNTP comprising EdU (5-ethynyldeoxyuridine). In some embodiments, the modification-sensitive sequencing comprises single-molecule real time (SMRT) sequencing and the at least one type of dNTP which comprises a modified base includes a dNTP comprising a 4mC, a dNTP comprising 5mC, a dNTP comprising 5hmC, a dNTP comprising 6 mA, and/or a dNTP comprising 8oxoG (8-oxodeoxyguanosine). In some embodiments, the at least one type of dNTP which comprises a modified base includes a dNTP comprising 4mC, a dNTP comprising 5mC, a dNTP comprising 5hmC, a dNTP comprising 6 mA, a dNTP comprising BrdU, dUTP, a dNTP comprising FldU, a dNTP comprising IdU, a dNTP comprising EdU, and/or a dNTP comprising 8oxoG.

In some embodiments, the modified base is 5mC or 5hmC. Analyzing the sequence data obtained in step (c) comprises the identification of regions comprising the modified base (e.g., 5mC or 5hmC) in non-CpG sequence contexts and classifying these regions as regions that were synthesized during the end repair. A modified base occurs in a non-CpG sequence context when a base other than G is immediately 3′ of the modified base. In some embodiments, the modified base is other than 5mC or 5hmC, a region of the one or more regions is defined as: (i) the sequence between two non-modified bases spanning a modified base, wherein the bases are of the same identity to the modified bases present in the at least one type of dNTP; and/or (ii) the sequence between a non-modified base and the end of a sequence read, wherein there is no additional non-modified bases between the non-modified base and the end of the sequence read, where the non-modified bases are the same identity as the modified base present in the at least one type of dNTP.

In some embodiments, the modified base is a methylated cytosine, such as 5mC or 5hmC, a region of the one or more regions is defined as: (i) the sequence between two non-methylated cytosines which span a methylated non-CpG cytosine; and/or (ii) the sequence between a non-methylated cytosine and the end of a sequence read wherein there is no additional non-methylated cytosine between the non-methylated cytosine and the end of the sequence read.

In some embodiments, the method further comprises: (i) filtering out sequence data from the one or more regions identified as being synthesized during the end repair such that these sequence data are not used for subsequent analysis; or (ii) flagging sequence data from the one or more regions identified as being synthesized during the end repair as potentially containing artifactual sequence data that may not be representative of the DNA sample. In some embodiments, the method further comprises analyzing at least some of the sequence data corresponding to regions that are not identified as being synthesized during the end repair to detect the presence or absence of base modifications or mutations present in the DNA sample. In some embodiments, the method is for detecting the methylation status of cytosines in the DNA sample, and wherein the analyzing the sequence data comprises filtering out the one or more regions of the end-repaired DNA that are identified as being synthesized during the end repair such that the one or more regions are not used to determine the methylation status of cytosines in the DNA sample. In some embodiments, the method is for detecting the single nucleotide variants (SNVs) in the DNA sample, and wherein the analyzing the sequence data comprises classifying all base calls within the one or more regions as not having double stranded support.

In some embodiments, the method further comprises quantifying the DNA damage in the DNA sample through the identification of the one or more regions of the end-repaired DNA that were synthesized during the end repair. In some embodiments, the level of DNA damage is used to predict whether or not a portion of the DNA in the DNA sample is derived from cancerous cells. In some embodiments, the DNA sample comprises cell-free DNA (cfDNA) and the method further comprises analyzing the sequence data obtained in step (c) to determine a level of measured artifacts in the cfDNA. In some embodiments, the method further comparing the level of measured artifacts to one or more reference levels. In some embodiments, the method further comprises (i) predicting whether or not a portion of the cfDNA is derived from cancerous cells using the level of measured artifacts and/or the comparison of the level of measured artifacts to one or more reference levels or (ii) determining a likelihood that a portion of the cfDNA is derived from cancerous cells using the level of measured artifacts and/or the comparison of the level of measured artifacts to one or more reference levels. In some embodiments, the DNA sample comprises cell-free DNA or DNA from formalin fixed paraffin embedded samples.

In another aspect, the present disclosure provides a method comprising: (a) subjecting the DNA sample to end repair to generate end-repaired DNA, wherein the end repair is performed with deoxynucleotide triphosphates (dNTPs) comprising 5mCTP; (b) performing a ligation reaction to ligate adapters to the end-repaired DNA to generate adapted DNA, wherein the ligation reaction also seals nicks present in the end-repaired DNA; (c) subjecting the adapted DNA to bisulfite sequencing to obtain sequencing data derived from the DNA sample; (d) analyzing the sequence data obtained in step (c) to identify one or more regions of the end-repaired DNA that were synthesized during the end repair by the presence of 5mC at non-CpG positions; and (e) optionally further comprising analyzing at least some of the sequence data corresponding to regions that are not identified as being synthesized during the end repair to detect the presence or absence of cytosine methylation in the DNA sample.

In another aspect, the present disclosure provides a method comprising: (a) subjecting the DNA sample to end repair to generate end-repaired DNA, wherein the end repair is performed with deoxynucleotide triphosphates (dNTPs), wherein at least one type of dNTP comprises a modified base; (b) performing a ligation reaction to ligate adapters to the end-repaired DNA to generate adapted DNA, wherein the ligation reaction also seals nicks present in the end-repaired DNA; (c) subjecting the adapted DNA to modification-sensitive sequencing to obtain sequencing data derived from the DNA sample, wherein the modification-sensitive sequencing is capable of identifying the base modification in the at least one type of dNTP, wherein the modification-sensitive sequencing is nanopore sequencing, single molecule real time sequencing or Tet-assisted pyridine borane sequencing (TAPS); (d) analyzing the sequence data obtained in step (c) to identify one or more regions of the end-repaired DNA that were synthesized during the end repair by the presence of the base modification in the at least one type of dNTP used in step (a); and (e) further comprising analyzing at least some of the sequence data corresponding to regions that are not identified as being synthesized during the end repair to detect the presence or absence of mutations present in the DNA sample.

In another aspect, the present disclosure provides a method comprising: (a) subjecting the DNA sample to end repair to generate end-repaired DNA, wherein the end repair is performed with deoxynucleotide triphosphates (dNTPs), wherein at least one type of dNTP comprises a modified base, wherein the end repair is performed with a DNA polymerase which does not have 5′-3′ exonuclease activity and/or is not a strand displacing DNA polymerase, such as T4 DNA polymerase, T7 DNA polymerase or Klenow fragment; (b) performing an A-tailing reaction as a single tube reaction with the end repair, optionally wherein the A-tailing is performed using a DNA polymerase that does not possess 3′-5′ exonuclease activity and/or is not a strand displacing DNA polymerase; (c) performing a ligation reaction to ligate adapters to the end-repaired A-tailed DNA to generate adapted DNA, wherein the ligation reaction also seals nicks present in the end-repaired DNA; (d) subjecting the adapted DNA to modification-sensitive sequencing to obtain sequencing data derived from the DNA sample, wherein the modification-sensitive sequencing is capable of identifying the base modification in the at least one type of dNTP; (e) analyzing the sequence data obtained in step (d) to identify one or more regions of the end-repaired DNA that were synthesized during the end repair by the presence of the base modification in the at least one type of dNTP used in step (a); and (f) optionally further comprising analyzing at least some of the sequence data corresponding to regions that are not identified as being synthesized during the end repair to detect the presence or absence of base modifications or mutations present in the DNA sample.

In another aspect, the present disclosure provides a method comprising: (a) subjecting the DNA sample to end repair to generate end-repaired DNA, wherein the end repair is performed with deoxynucleotide triphosphates (dNTPs), wherein at least one type of dNTP comprises a modified base, wherein the end repair is performed with a DNA polymerase which does not have 5′-3′ exonuclease activity and/or is not a strand displacing DNA polymerase, such as T4 DNA polymerase, T7 DNA polymerase or Klenow fragment; (b) performing a reaction cleanup step on the end-repaired DNA followed by an A-tailing reaction to generate A-tailed DNA; (c) performing a ligation reaction to ligate adapters to the A-tailed DNA to generate adapted DNA, wherein the ligation reaction also seals nicks present in the end-repaired DNA; (d) subjecting the adapted DNA to modification-sensitive sequencing to obtain sequencing data derived from the DNA sample, wherein the modification-sensitive sequencing is capable of identifying the base modification in the at least one type of dNTP; (e) analyzing the sequence data obtained in step (d) to identify one or more regions of the end-repaired DNA that were synthesized during the end repair by the presence of the base modification in the at least one type of dNTP used in step (a); and (f) optionally further comprising analyzing at least some of the sequence data corresponding to regions that are not identified as being synthesized during the end repair to detect the presence or absence of base modifications or mutations present in the DNA sample.

In another aspect, the present disclosure provides a method comprising: (a) subjecting the DNA sample to end repair to generate end-repaired DNA, wherein the end repair is performed with deoxynucleotide triphosphates (dNTPs), wherein at least one type of dNTP comprises a modified base wherein the end repair is performed with a DNA polymerase which does not have 5′-3′ exonuclease activity and/or is not a strand displacing DNA polymerase, such as T4 DNA polymerase, T7 DNA polymerase or Klenow fragment; (b) performing a blunt-end ligation reaction to ligate adapters to the end-repaired DNA to generate adapted DNA, wherein the ligation reaction also seals nicks present in the end-repaired DNA; (c) subjecting the adapted DNA to modification-sensitive sequencing to obtain sequencing data derived from the DNA sample, wherein the modification-sensitive sequencing is capable of identifying the base modification in the at least one type of dNTP; (d) analyzing the sequence data obtained in step (c) to identify one or more regions of the end-repaired DNA that were synthesized during the end repair by the presence of the base modification in the at least one type of dNTP used in step (a); and (e) optionally further comprising analyzing at least some of the sequence data corresponding to regions that are not identified as being synthesized during the end repair to detect the presence or absence of base modifications or mutations present in the DNA sample.

In some embodiments, a kit is provided comprising a) a first reagent for end repair to generate end-repaired DNA, wherein the first reagent comprises at least one type of dNTP that comprises a modified base, b) a second reagent for ligating adapters to the end-repaired DNA to generate adapted DNA, wherein the second reagent also seals nicks present in the end-repaired DNA, c) a reagent for modification-sensitive sequencing that is capable of identifying the base modification in the at least one type of dNTP and/or a DNA polymerase for incorporating the first reagent into DNA during end repair, and d) library adaptors having distinct molecular barcodes.

In some embodiments, the kit further comprises a plurality of oligonucleotide probes and/or primers for sequencing. In some embodiments, the first reagent of the kit comprises at least one type of dNTP that comprises a modified base selected from a dNTP comprising 4-methylcytosine (4mC), a dNTP comprising 5-methylcytosine (5mC), a dNTP comprising 5-hydroxymethyl-cytosine (5hmC), a dNTP comprising N6-methyladenosine (6 mA), a dNTP comprising bromodeoxyuridine (BrdU), a dNTP comprising 8-oxoguanine (8oxoG), dUTP, a dNTP comprising fluorodeoxyuridine (FldU), a dNTP comprising iododeoxyuridine (IdU), and/or a dNTP comprising ethynyldeoxyuridine (EdU). In some embodiments, the DNA polymerase of the kit does not have 5′-3′ exonuclease activity and/or is not a strand displacing DNA polymerase. In some embodiments, the DNA polymerase of the kit is T4 DNA polymerase, T7 DNA polymerase or Klenow fragment. In some embodiments, the DNA polymerase of the kit has 5′-3′ exonuclease activity and/or is a strand displacing DNA polymerase. In some embodiments, the kit further comprises a reagent for performing an A-tailing reaction. In some embodiments, the reagent for performing the A-tailing reaction comprises a DNA polymerase that does not possess 5′-3′ exonuclease activity and/or is not a strand displacing DNA polymerase, optionally the reagent for performing the A-tailing reaction is HemoKlen Taq. In some embodiments, the reagent for performing the A-tailing reaction comprises a Taq DNA polymerase, Tfl DNA Polymerase, Bst DNA Polymerase, Large Fragment or Tth DNA polymerase. In some embodiments, the reagent for performing the A-tailing reaction comprises a DNA polymerase that does not possess 3′-5′ exonuclease activity, optionally wherein the reagent for performing the A-tailing reaction is Klenow Fragment lacking 3′-5′ exonuclease activity. In some embodiments, the reagent for performing the A-tailing reaction comprises a DNA polymerase that has 5′-3′ exonuclease activity and/or is a strand displacing DNA polymerase.

In some embodiments, the kit comprises a) the plurality of oligonucleotide probes selectively hybridize to at least 5, 6, 7, 8, 9, 10, 20, 30, 40 or all genes selected from ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1, b) the library adaptors do not include flow cell sequences or sequences that permit the formation of hairpin loops for sequencing, c) the library adaptors are blunt ended and Y-shaped, and/or d) the library adaptors are less than or equal to 40 nucleic acid bases in length. In some embodiments, the kit further comprises instructions for performing any of the methods described herein.

The embodiments described herein include, but are not limited to, the following.

Embodiment 1 is a method comprising:
(a) subjecting a DNA sample to end repair to generate end-repaired DNA, wherein the end repair is performed with deoxynucleotide triphosphates (dNTPs), wherein at least one type of dNTP comprises a modified base;
(b) performing a ligation reaction to ligate adapters to the end-repaired DNA to generate adapted DNA, wherein the ligation reaction also seals nicks present in the end-repaired DNA;
(c) subjecting the adapted DNA to modification-sensitive sequencing to obtain sequencing data derived from the DNA sample, wherein the modification-sensitive sequencing is capable of identifying the base modification in the at least one type of dNTP; and
(d) analyzing the sequence data obtained in step (c) to identify one or more regions of the end-repaired DNA that were synthesized during the end repair by the presence of the base modification in the at least one type of dNTP used in step (a).

Embodiment 2 is the method of embodiment 1, wherein the end repair is performed with a DNA polymerase which does not have 5′-3′ exonuclease activity and/or is not a strand displacing DNA polymerase.

Embodiment 3 is the method of embodiment 2, wherein the DNA polymerase is T4 DNA polymerase, T7 DNA polymerase or Klenow fragment.

Embodiment 4 is the method of embodiment 1, wherein the end repair is performed with a DNA polymerase which has 5′-3′ exonuclease activity and/or is a strand displacing DNA polymerase.

Embodiment 5 is the method of any one of embodiments 1 to 4, wherein the at least one type of dNTP which comprises a modified base modified base includes a dNTP comprising 4-methylcytosine (4mC), a dNTP comprising 5-methylcytosine (5mC), a dNTP comprising 5-hydroxymethyl-cytosine (5hmC), a dNTP comprising N6-methyladenosine (6 mA), a dNTP comprising bromodeoxyuridine (BrdU) and/or a dNTP comprising 8-oxoguanine (8oxoG).

Embodiment 6 is the method of any one of embodiments 1 to 5, further comprising between steps (a) and (b) performing an A-tailing reaction.

Embodiment 7 is the method of embodiment 6, wherein the end-repair and the A-tailing reaction are performed in the same reaction mixture, optionally wherein the end-repair and the A-tailing reaction are performed a single tube and/or optionally wherein the end-repair and the A-tailing reaction are performed without an intervening clean-up step.

Embodiment 8 is the method of embodiment 7, wherein the A-tailing is performed using a DNA polymerase that does not possess 5′-3′ exonuclease activity and/or is not a strand displacing DNA polymerase, optionally wherein the DNA polymerase is HemoKlen Taq.

Embodiment 9 is the method of embodiment 7, wherein the A-tailing is performed using a thermostable DNA polymerase.

Embodiment 10 is the method of embodiment 7, wherein the A-tailing is performed using Taq DNA polymerase, Tfl DNA Polymerase, Bst DNA Polymerase, Large Fragment or Tth DNA polymerase.

Embodiment 11 is the method of embodiment 6, wherein the end-repair and the A-tailing reaction are performed as separate reactions, wherein a reaction clean-up step is performed after the end-repair and before the A-tailing reaction.

Embodiment 12 is the method of embodiment 11, wherein the reaction clean-up step removes unincorporated dNTPs.

Embodiment 13 is the method of embodiment 11 or 12, wherein the A-tailing is performed using a DNA polymerase that does not possess 3′-5′ exonuclease activity, optionally wherein the DNA polymerase is Klenow Fragment lacking 3′-5′ exonuclease activity.

Embodiment 14 is the method of embodiment 7 or embodiment 11, wherein the A-tailing is performed using a DNA polymerase that possesses 5′-3′ exonuclease activity and/or is a strand displacing DNA polymerase.

Embodiment 15 is the method of any one of embodiments 6-14, wherein the A tailing reaction is performed at a higher temperature than the end repair, optionally wherein the end repair is performed at about 15-35° C. and/or the A tailing is performed at a temperature over about 60° C., further optionally wherein the temperature over 60° C. is about 60° C.-75° C.

Embodiment 16 is the method of any one of embodiments 1-5, wherein the ligation reaction is a blunt-end ligation reaction.

Embodiment 17 is the methods of any one of embodiments 6-15, wherein the ligation reaction is a sticky-end ligation reaction.

Embodiment 18 is the method of any one of embodiments 1-17, wherein the modification-sensitive sequencing comprises a conversion procedure that changes the base pairing specificity of the base or does not change the base pairing specificity of the base, depending on the modification status of the base.

Embodiment 19 is the method of embodiment 18, wherein the modified base is a methylated cytosine and wherein the conversion procedure converts methylated cytosines, for example wherein the conversion procedure is Tet-assisted conversion with a substituted borane reducing agent, optionally wherein the substituted borane reducing agent is 2-picoline borane, borane pyridine, tert-butylamine borane, or ammonia borane.

Embodiment 20 is the method of embodiment 18, wherein the modified base is a methylated cytosine and wherein the conversion procedure converts unmethylated cytosines, for example wherein the conversion procedure is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq) or single-enzyme 5-methylctyosine sequencing (SEM-seq).

Embodiment 21 is the method of any one of embodiments 1-17, wherein the modification-sensitive sequencing comprises nanopore-based sequencing or single-molecule real time (SMRT) sequencing.

Embodiment 22 is the method of embodiment 21, wherein the modification-sensitive sequencing comprises nanopore-based sequencing and wherein the at least one type of dNTP which comprises a modified base includes a dNTP comprising 4mC, a dNTP comprising 5mC, a dNTP comprising 5hmC, a dNTP comprising 6 mA, a dNTP comprising BrdU, dUTP, a dNTP comprising FldU, a dNTP comprising IdU, and/or a dNTP comprising EdU.

Embodiment 23 is the method of embodiment 21, wherein the modification-sensitive sequencing comprises single-molecule real time (SMRT) sequencing and wherein the at least one type of dNTP which comprises a modified base includes a dNTP comprising a 4mC, a dNTP comprising 5mC, a dNTP comprising 5hmC, a dNTP comprising 6 mA, and/or a dNTP comprising 8oxoG.

Embodiment 24 is the method of any one of embodiments 1-23, wherein the modified base is 5mC.

Embodiment 25 is the method of any one of embodiments 1-23, wherein the modified base is 5hmC.

Embodiment 26 is the method of any one of embodiments 1-25, wherein analyzing the sequence data obtained in step (c) comprises the identification of regions comprising the modified base in non-CpG sequence contexts and classifying these regions as regions that were synthesized during the end repair.

Embodiment 27 is the method of any one of embodiments 1-23, wherein the modified base is other than 5mC or 5hmC, and a region of the one or more regions is defined as:
(i) the sequence between two non-modified bases spanning a modified base, wherein the bases are of the same identity to the modified bases present in the at least one type of dNTP; and/or
(ii) the sequence between a non-modified base and the end of a sequence read, wherein there is no additional non-modified bases between the non-modified base and the end of the sequence read, where the non-modified bases are the same identity as the modified base present in the at least one type of dNTP.

Embodiment 28 is the method of any one of embodiments 1-23, wherein the modified base is a methylated cytosine, such as 5mC or 5hmC, a region of the one or more regions is defined as:
(i) the sequence between two non-methylated cytosines which span a methylated non-CpG cytosine; and/or
(ii) the sequence between a non-methylated cytosine and the end of a sequence read wherein there is no additional non-methylated cytosine between the non-methylated cytosine and the end of the sequence read.

Embodiment 29 is the method of any one of embodiments 1-28, further comprising: (i) filtering out sequence data from the one or more regions identified as being synthesized during the end repair such that these sequence data are not used for subsequent analysis; or (ii) flagging sequence data from the one or more regions identified as being synthesized during the end repair as potentially containing artifactual sequence data that may not be representative of the DNA sample.

Embodiment 30 is the method of any one of embodiments 1-29, further comprising analyzing at least some of the sequence data corresponding to regions that are not identified as being synthesized during the end repair to detect the presence or absence of base modifications or mutations present in the DNA sample.

Embodiment 31 is the method of any one of embodiments 1-30, wherein the method is for detecting the methylation status of cytosines in the DNA sample, and wherein the analyzing the sequence data comprises filtering out the one or more regions of the end-repaired DNA that are identified as being synthesized during the end repair such that the one or more regions are not used to determine the methylation status of cytosines in the DNA sample.

Embodiment 32 is the method of any one of embodiments 1-31, wherein the method is for detecting the single nucleotide variants (SNVs) in the DNA sample, and wherein the analyzing the sequence data comprises classifying all base calls within the one or more regions as not having double stranded support.

Embodiment 33 is the method of any one of embodiments 1-32, wherein the method further comprises quantifying the DNA damage in the DNA sample through the identification of the one or more regions of the end-repaired DNA that were synthesized during the end repair.

Embodiment 34 is the method of embodiment 33, wherein the level of DNA damage is used to predict whether or not a portion of the DNA in the DNA sample is derived from cancerous cells.

Embodiment 35 is the method of any one of embodiments 1-34, wherein the DNA sample comprises cell-free DNA (cfDNA) and the method further comprises analyzing the sequence data obtained in step (c) to determine a level of measured artifacts in the cfDNA.

Embodiment 36 is the method of embodiment 35, further comprising comparing the level of measured artifacts to one or more reference levels.

Embodiment 37 is the method of embodiment 35 or 36, further comprising (i) predicting whether or not a portion of the cfDNA is derived from cancerous cells using the level of measured artifacts and/or the comparison of the level of measured artifacts to one or more reference levels or (ii) determining a likelihood that a portion of the cfDNA is derived from cancerous cells using the level of measured artifacts and/or the comparison of the level of measured artifacts to one or more reference levels.

Embodiment 38 is the method of any one of embodiments 1-34, wherein the DNA sample comprises cell-free DNA or DNA from formalin fixed paraffin embedded samples.

Embodiment 39 is the method of the immediately preceding embodiment, wherein the DNA sample comprises cell-free DNA.

Embodiment 40 is the method of any one of the preceding embodiments, wherein the sample is from a subject and the method further comprises determining the presence or absence of cancer in the subject based at least in part on the sequencing data.

Embodiment 41 is the method of any one of the preceding embodiments, further comprising at least one DNA amplification step, optionally wherein the DNA amplification step is performed after step (b) and before step (c).

Embodiment 42 is the method of the immediately preceding embodiment, wherein the DNA amplification step comprises PCR.

Embodiment 43 is the method of any one of the preceding embodiments, wherein the adapters comprise molecular barcodes.

Embodiment 44 is the method of any one of the preceding embodiments, further comprising enriching the DNA for a plurality of target regions prior to step (c).

Embodiment 45 is the method of embodiment 44, wherein the plurality of target regions comprise epigenetic target regions.

Embodiment 46 is the method of embodiment 45, wherein the epigenetic target regions comprise hypermethylation variable target regions.

Embodiment 47 is the method of embodiment 45 or 46, wherein the epigenetic target regions comprise hypomethylation variable target regions.

Embodiment 48 is the method of any one of embodiments 44-47, wherein the plurality of target regions comprise sequence-variable target regions.

Embodiment 49 is a kit comprising:
a) a first reagent for end repair to generate end-repaired DNA, wherein the first reagent comprises at least one type of dNTP that comprises a modified base;
b) a second reagent for ligating adapters to the end-repaired DNA to generate adapted DNA, wherein the second reagent also seals nicks present in the end-repaired DNA;
c) a reagent for modification-sensitive sequencing that is capable of identifying the base modification in the at least one type of dNTP and/or a DNA polymerase for incorporating the first reagent into DNA during end repair; and
d) library adaptors having distinct molecular barcodes.

Embodiment 50 is the kit of embodiment 49, further comprising a plurality of oligonucleotide probes and/or primers for sequencing.

Embodiment 51 is the kit of embodiment 49 or 50, wherein the first reagent comprises at least one type of dNTP that comprises a modified base selected from a dNTP comprising 4-methylcytosine (4mC), a dNTP comprising 5-methylcytosine (5mC), a dNTP comprising 5-hydroxymethyl-cytosine (5hmC), a dNTP comprising N6-methyladenosine (6 mA), a dNTP comprising bromodeoxyuridine (BrdU), a dNTP comprising 8-oxoguanine (8oxoG), dUTP, a dNTP comprising fluorodeoxyuridine (FldU), a dNTP comprising iododeoxyuridine (IdU), and/or a dNTP comprising ethynyldeoxyuridine (EdU).

Embodiment 52 is the kit of any one of embodiments 49-51, wherein the DNA polymerase does not have 5′-3′ exonuclease activity and/or is not a strand displacing DNA polymerase.

Embodiment 53 is the kit of any one of embodiments 49-52, wherein the DNA polymerase is T4 DNA polymerase, T7 DNA polymerase or Klenow fragment.

Embodiment 54 is the kit of embodiment 49, wherein the DNA polymerase has 5′-3′ exonuclease activity and/or is a strand displacing DNA polymerase.

Embodiment 55 is the kit of any one of embodiments 49-54, further comprising a reagent for performing an A-tailing reaction.

Embodiment 56 is the kit of the immediately preceding embodiment, further comprising a reagent for a clean-up step following the A-tailing reaction, optionally wherein the reagent for the clean-up step is for removing unincorporated dNTPs.

Embodiment 57 is the kit of embodiment 55 or 56, wherein the reagent for performing the A-tailing reaction comprises a DNA polymerase that does not possess 5′-3′ exonuclease activity and/or is not a strand displacing DNA polymerase, optionally wherein the reagent for performing the A-tailing reaction is HemoKlen Taq.

Embodiment 58 is the kit of any one of embodiments 55-57, wherein the reagent for performing the A-tailing reaction comprises a Taq DNA polymerase, Tfl DNA Polymerase, Bst DNA Polymerase, Large Fragment or Tth DNA polymerase.

Embodiment 59 is the kit of any one of embodiments 55-58, wherein the reagent for performing the A-tailing reaction comprises a thermostable DNA polymerase.

Embodiment 60 is the kit of any one of embodiments 55-56, wherein the reagent for performing the A-tailing reaction comprises a DNA polymerase that does not possess 3′-5′ exonuclease activity, optionally wherein the reagent for performing the A-tailing reaction is Klenow Fragment lacking 3′-5′ exonuclease activity.

Embodiment 61 is the kit of any one of embodiments 55-56, wherein the reagent for performing the A-tailing reaction comprises a DNA polymerase that has 5′-3′ exonuclease activity and/or is a strand displacing DNA polymerase.

Embodiment 62 is the kit of any one of embodiments 49-61, wherein:
a) the plurality of oligonucleotide probes selectively hybridize to at least 5, 6, 7, 8, 9, 10, 20, 30, 40 or all genes selected from ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1;
b) the library adaptors do not include flow cell sequences or sequences that permit the formation of hairpin loops for sequencing;
c) the library adaptors are blunt ended and Y-shaped; and/or
d) the library adaptors are less than or equal to 40 nucleic acid bases in length.

Embodiment 63 is the kit of any one of embodiments 49-62, further comprising instructions for performing the method of any one of embodiments 1-48.

In some embodiments, the results of the methods disclosed herein are used as an input to generate a report. The report may be in a paper or electronic format. For example the true methylation status of cytosines or variants, as obtained by the methods disclosed herein, or information derived therefrom, can be displayed directly in such a report. Alternatively or additionally, diagnostic information or therapeutic recommendations which are at least in part based on the methods disclosed herein can be included in the report.

The various steps of the methods disclosed herein may be carried out at the same or different times, in the same or different geographical locations, e.g. countries, and/or by the same or different people.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

In FIGS. 1-6, solid mushrooms represent methylated CpG sites and open mushrooms represent unmethylated CpG sites. Solid thick lines represent regions of the DNA molecule that were present in the original DNA molecule (i.e. prior to end repair). Dashed lines represent synthesized regions synthesized during end repair, including regions added to the 3′end of DNA molecules at 5′ overhangs during end repair, and internal synthesized regions which result from gap filling and nick translation. Solid thin lines represent synthesized regions synthesized during A-tailing. Cross-hatched circles represent the polymerases used in end repair. Open circles represent the polymerases used in A tailing. Triangles represent ligation sites. Stars represent 5mCpH sites.

FIG. 1 is a schematic which shows the effect of combined end repair and A-tailing on the methylation status of cytosines in DNA molecules when using unmodified dCTP in the end repair.

FIG. 2 is a schematic which shows the effect of separate end repair and A-tailing reactions on the methylation status of intact, nicked and gapped DNA molecules when using unmodified dCTP in the end repair.

FIG. 3 is a schematic which shows the effect of end repair followed by blunt end ligation on the methylation status of intact, nicked and gapped DNA molecules when using unmodified dCTP in the end repair.

FIG. 4 is a schematic which shows the effect of combined end repair and A-tailing reactions on the methylation status of intact, nicked and gapped DNA molecules when using methylated CTP in the end repair and A tailing reaction.

FIG. 5 is a schematic which shows the effect of separate end repair and A-tailing reactions on the methylation status of intact, nicked and gapped DNA molecules when using methylated CTP in the end repair.

FIG. 6 is a schematic which shows the effect of end repair followed by blunt end ligation on the methylation status of intact, nicked and gapped DNA molecules when using methylated CTP in the end repair.

FIG. 7A is a plot showing the estimated level of end repair in paired strands of a DNA molecule. FIG. 7B is a sequence diagram which shows exemplary aligned forward (bottom two sequences) and reverse strands (top two sequences). Aligned reads are shown, with ‘.’ indicating any non-cytosine base, ‘o’ and ‘M’ indicating unmethylated and methylated cytosines, respectively.

FIG. 8 shows a series of M-bias plots, which show percentage methylation levels at CpG sites (upper row) and non-CpG sites (lower row) throughout sequence reads. The two left hand columns represent experiments performed using a single tube end repair/A-tailing reaction with dCTP comprising unmodified cytosine, whereas the two right hand columns represent experiments performed using a single tube end repair/A-tailing reaction with dCTP comprising 5-methylcytosine (5mCTP).

FIG. 9 shows a series of M-bias plots, which show percentage methylation levels at CpG sites (upper row) and non-CpG sites (lower row) throughout sequence reads. The two left hand columns represent experiments performed using end repair with dCTP comprising unmodified cytosine, followed by blunt end ligation. The three right hand columns represent experiments performed using a single tube end repair/A-tailing reaction with dCTP comprising unmodified cytosine followed by sticky end ligation.

FIG. 10 shows a series of M-bias plots, which show percentage methylation levels at CpG sites (upper row) and non-CpG sites (lower row) throughout sequence reads. The three left hand columns represent experiments performed using end repair with dCTP comprising 5-methylcytosine (5mCTP), followed by blunt end ligation. The three right hand columns represent experiments performed using a single tube end repair/A-tailing reaction with dCTP comprising 5-methylcytosine (5mCTP) followed by sticky end ligation.

FIGS. 11A-11B show a comparison of incorporation of mCTP in positive (A) and negative (B) control regions versus incorporation of CTP after end-repair/A-tailing.

FIGS. 12A-12D show comparisons of mCTP end trimming versus CTP end trimming after end-repair/A-tailing.

FIG. 13 is a schematic diagram of an example of a system suitable for use with some embodiments of the disclosure.

### DETAILED DESCRIPTION

Reference will now be made in detail to certain embodiments of the disclosure. While the disclosure will be described in conjunction with such embodiments, it will be understood that they are not intended to limit the disclosure to those embodiments. On the contrary, the disclosure is intended to cover all alternatives, modifications, and equivalents, which may be included within the disclosure as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids.

Numeric ranges are inclusive of the numbers defining the range. Measured and measurable values are understood to be approximate, taking into account significant digits and the error associated with the measurement.

Unless specifically noted in the above specification, embodiments in the specification that recite “comprising” various components are also contemplated as “consisting of” or “consisting essentially of” the recited components.

The section headings used herein are for organizational purposes and are not to be construed as limiting the disclosed subject matter in any way.

All patents, patent applications, websites, other publications or documents and the like cited herein whether supra or infra, are expressly incorporated by reference in their entirety for all purposes to the same extent as if each individual item were specifically and individually indicated to be so incorporated by reference. If different versions of a publication, website or the like are published at different times, the version most recently published at the effective filing date of the application is meant, unless otherwise indicated.

### Definitions

A “reaction cleanup” refers to the removal of contaminants such as salts, enzymes, unincorporated dNTPs, primers, ethidium bromide, and other impurities that can interfere with downstream analysis. For example, when a reaction cleanup is performed between end repair and an A-tailing reaction, it removes unincorporated dNTPs such that the A-tailing reaction can be performed solely in the presence of dATP (i.e. not dCTP, dGTP and dCTP, as used in the end tailing reaction). Reaction cleanups can be performed using commercially available kits such as MinElute Reaction Cleanup Kit (Qiagen)

“Synthesized regions”, also referred to as “regions of the end-repaired DNA that were synthesized during the end repair” refer to regions of the DNA that were not present in the DNA prior to the end repair and A-tailing reactions. They are regions which have been synthesized by the polymerases used in the end repair and/or A tailing reactions, if present. In instances where the A-tailing is performed in the same tube as the end repair reaction, all four types of dNTPs will be present, and thus the polymerases used for A-tailing may generate synthesized regions, e.g. through nick translation. In instances where the A-tailing is performed separately to the end repair reaction, and these steps are separated by a reaction cleanup, only dATP will be present in the A-tailing reaction, and thus the polymerases used for A-tailing will not typically generate synthesized regions because the dNTP components are not all present in the A-tailing reaction mix.

As used herein, “base pairing specificity” refers to the standard DNA base (A, C, G, or T) for which a given base most preferentially pairs. Thus, for example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G, for example, is irrelevant because uracil nonetheless most preferentially pairs with A among the four standard DNA bases.

A “type of dNTP” refers to a dNTP comprising a specific base, including A, T, G or C. Accordingly, wherein an end repair reaction is performed with dNTPs, wherein at least one type of dNTP comprises a modified base, the end repair reaction may be performed using dCTP comprising 5mC, and dATP, dTTP and dGTP all comprising non-modified bases.

“Capable of identifying the base modification in the at least one type of dNTP” refers to the ability of a modification-sensitive sequencing method to detect the presence or absence of the base modification in the at least one type of dNTP comprising a modified base used in the end repair. This detection of the base modification may be direct, such as in nanopore sequencing or single molecule real time sequencing, wherein the sequencing data itself indicates the presence or absence of a base modification. Alternatively, the detection of the base modification may be indirect, for example wherein the method involves a conversion procedure which alters the base pairing specificity dependent on the base modification status. It is these changes in base pairing specificity which can be detected by the sequencing method, e.g. through the comparison of the sequencing data to a reference sequence. Moreover, a modification-sensitive sequencing method is capable of identifying the base modification in the at least one type of dNTP regardless of whether it can distinguish one base modification from all other base modifications. For example, one form of modification-sensitive sequencing is sequencing after bisulfite conversion. This method is capable of distinguishing 5hmC and 5mC from unmethylated cytosine, but cannot distinguish 5hmC from 5mC.

Bases of the “same identity” refer to the same base, regardless of modification status of that base. For example, cytosine is considered to be the “same identity” as 5-methylcytosine (5mC) and/or 5-hydroxymethyl-cytosine (5hmC), despite them having different modification statuses.

“Capturing” one or more target nucleic acids refers to preferentially isolating or separating the one or more target nucleic acids from non-target nucleic acids.

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) into a fluid found in the organism, and may be obtained from a sample of the fluid without the need to perform an in vitro cell lysis step.

As used herein, “cellular nucleic acids” means nucleic acids that are disposed within one or more cells from which the nucleic acids have originated, at least at the point a sample is taken or collected from a subject, even if those nucleic acids are subsequently removed (e.g., via cell lysis) as part of a given analytical process.

DNA is “derived from cancerous cells” if it originated from a tumor cell. Cell free DNA derived from cancerous cells includes ctDNA or circulating tumor DNA. Tumor cells are neoplastic cells that originated from a tumor, regardless of whether they remain in the tumor or become separated from the tumor (as in the cases, e.g., of metastatic cancer cells and circulating tumor cells).

The term “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′-3′ direction of the nucleic acid sequence)). In some embodiments, DNA methylation refers to addition of a methyl group to adenine, such as in N⁶-methyladenine (6 mA). In some embodiments, DNA methylation is 5-methylation (modification of the carbon in the 5th position of the cytosine ring). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the carbon in the 3^{rd} position of the cytosine ring). In some embodiments, 3C methylation comprises addition of a methyl group to the 3C position of the cytosine to generate 3-methylcytosine (3mC). Methylation can also occur at non-CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The “modified nucleoside profile of DNA” means the position and identity of the nucleoside and the modification status of the nucleoside, such as methylations, within a DNA sequence. As described above, different modification sensitive sequencing methods can be used to detect such modifications. This includes methods which involve conversion followed by sequencing detect one or more different types of modified or unmodified nucleoside. For example, the TAPS method detects, but does not distinguish between, 5-methylcytosine (5mC) and 5-hydroxymethyl-cytosine (5hmC). Hence, a method for analyzing the modified nucleoside profile of DNA in a sample typically means identifying particular modifications or groups of modification, such as 5mC and/or 5hmC. Modified nucleosides are identified according to the specific method/conversion procedure being used as described above. This generally involves comparing sequence data obtained from DNA that has been subjected to a conversion procedure to a reference sequence. Typically, the method involves (i) comparing the sequence data with (A) one or more pre-determined reference sequence; or (B) sequence data obtained by sequencing a sub-sample of the DNA that was not subjected to the conversion procedure, for example a subsample that was separated before subjecting a separate subsample to the conversion procedure, for example as described herein; and (ii) identifying point differences between the converted DNA sequences and the reference sequence(s) (A) or non-converted DNA sequences (B) as nucleosides (in the initial sample) having a modification status that permits a change in base pairing specificity on exposure to the conversion procedure.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, “without substantially altering base-pairing specificity” of a given nucleobase means that a majority of molecules comprising that nucleobase that can be sequenced do not have alterations of the base pairing specificity of the second nucleobase relative to its base pairing specificity as it was in the originally isolated sample. In some embodiments, 75%, 90%, 95%, or 99% of molecules comprising that nucleobase that can be sequenced do not have alterations of the base pairing specificity of the second nucleobase relative to its base pairing specificity as it was in the originally isolated sample.

As used herein, “base pairing specificity” refers to the standard DNA base (A, C, G, or T) for which a given base most preferentially pairs. Thus, for example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, “modified cytosine” refers to a cytosine in which at least one position of the cytosine has been substituted with a chemical moiety, such as a methyl or hydroxymethyl, that is different from the substituent at that position in unmodified cytosine. For the avoidance of doubt, “modified cytosine” does not include unmodified cytosine.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

“Capturing” one or more target nucleic acids refers to preferentially isolating or separating the one or more target nucleic acids from non-target nucleic acids.

A “captured set” of nucleic acids refers to nucleic acids that have undergone capture.

A “target-region set” or “set of target regions” refers to a plurality of genomic loci targeted for capture and/or targeted by a set of probes (e.g., through sequence complementarity).

“Corresponding to a target region set” means that a nucleic acid, such as cfDNA, originated from a locus in the target region set or specifically binds one or more probes for the target-region set.

As used herein, a “differentially methylated region” (DMR) refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a DMR has a detectably higher degree of methylation (e.g., a hypermethylated region) in at least one cell or tissue type relative to the degree of methylation in the same region of DNA from at least one other cell or tissue type or from the same cell or tissue type from a healthy subject. In some embodiments, a DMR has a detectably lower degree of methylation (e.g., a hypomethylated region) in at least one cell or tissue type relative to the degree of methylation in the same region of DNA from at least one other cell or tissue type or from the same cell or tissue type from a healthy subject.

“Specifically binds” in the context of an probe or other oligonucleotide and a target sequence means that under appropriate hybridization conditions, the oligonucleotide or probe hybridizes to its target sequence, or replicates thereof, to form a stable probe:target hybrid, while at the same time formation of stable probe:non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, N Y, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Sequence-variable target region set” refers to a set of target regions that may exhibit changes in sequence such as nucleotide substitutions (i.e., single nucleotide variations), insertions, deletions, or gene fusions or transpositions in neoplastic cells (e.g., tumor cells and cancer cells).

“Epigenetic target region set” refers to a set of target regions that may show sequence-independent changes in neoplastic cells (e.g., tumor cells and cancer cells) or that may show sequence-independent changes in cfDNA from subjects having cancer relative to cfDNA from healthy subjects. Examples of sequence-independent changes include, but not limited to, changes in methylation (increases or decreases), nucleosome distribution, CTCF binding, transcription start sites, and regulatory protein binding regions. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

As used herein, “methylation status” can refer to the presence or absence of methyl group on a DNA base (e.g. cytosine) at a particular genomic position in a nucleic acid molecule. It can also refer to the degree of methylation in a nucleic acid sequence (e.g., highly methylated, low methylated, intermediately methylated or unmethylated nucleic acid molecules). The methylation status can also refer to the number of nucleotides methylated in a particular nucleic acid molecule.

As used herein, “mutation” refers to a variation from a known reference sequence and includes mutations such as, for example, single nucleotide variants (SNVs), and insertions or deletions (indels). A mutation can be a germline or somatic mutation. In some embodiments, a reference sequence for purposes of comparison is a wildtype genomic sequence of the species of the subject providing a test sample, typically the human genome.

As used herein, the terms “neoplasm” and “tumor” are used interchangeably. They refer to abnormal growth of cells in a subject. A neoplasm or tumor can be benign, potentially malignant, or malignant. A malignant tumor is referred to as a cancer or a cancerous tumor.

As used herein, “next-generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules. Examples of commercially available instruments for performing next-generation sequencing include, but are not limited to, NextSeq, HiSeq, NovaSeq, MiSeq, Ion PGM and Ion GeneStudio S5.

As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), distinguish nucleic acids from different partitions (e.g., representing a partition tag) or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode. Terms such as “library adaptors having distinct molecular barcodes” encompass library adaptors for uniquely or non-uniquely tagging molecules, in that regardless of whether the adaptors are for unique or non-unique tagging, distinct barcodes will be present in the population of adaptors.

As used herein, DNA that is “not immobilized” or that is “free in solution” refers to DNA that is not bound covalently or non-covalently to a solid support, such as a bead. Such DNA may be free in solution during any step (such as all steps) of the disclosed methods.

As used herein, “partitioning” refers to physically separating or fractionating a mixture of nucleic acid molecules in a sample based on a characteristic of the nucleic acid molecules. The partitioning can be physical partitioning of molecules. Partitioning can involve separating the nucleic acid molecules into groups or sets based on the level of epigenetic feature (for e.g., methylation). For example, the nucleic acid molecules can be partitioned based on the level of methylation of the nucleic acid molecules. In some embodiments, the methods and systems used for partitioning may be found in PCT Patent Application No. PCT/US2017/068329, which is hereby incorporated by reference in its entirety.

As used herein, “partitioned set” or “partition” refers to a set of nucleic acid molecules partitioned into a set or group based on the differential binding affinity of the nucleic acid molecules or proteins associated with the nucleic acid molecules to a binding agent. A partitioned set may also be referred to as a subsample. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level or degree of epigenetic feature (for e.g., methylation). For example, the nucleic acid molecules can be partitioned into three sets—one set for highly methylated nucleic acid molecules (first subsample, hyper partition, hyper partitioned set or hypermethylated partitioned set), a second set for low methylated nucleic acid molecules (second subsample, hypo partition, hypo partitioned set or hypomethylated partitioned set), and a third set for intermediate methylated nucleic acid molecules (third subsample, intermediate partitioned set, intermediately methylated partitioned set, residual partitioned set, or residual partition). In another example, the nucleic acid molecules can be partitioned based on the number of methylated nucleotides—one partitioned set can have nucleic acid molecules with nine methylated nucleotides, and another partitioned set can have unmethylated nucleic acid molecules (zero methylated nucleotides).

As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by inter-nucleosidic linkages. Typically, a polynucleotide comprises at least three nucleosides. Oligonucleotides often range in size from a few monomeric units, e.g., 3-4, to hundreds of monomeric units. Whenever a polynucleotide is represented by a sequence of letters, such as “ATGCCTG”, the nucleotides are in 5′-3′ order from left to right, and in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases.

As used herein, “processing” refers to a set of steps used to generate a library of nucleic acids that is suitable for sequencing. The set of steps can include, but are not limited to, partitioning, end repairing, addition of sequencing adapters, tagging, and/or PCR amplification of nucleic acids.

As used herein, “quantitative measure” refers to an absolute or relative measure. A quantitative measure can be, without limitation, a number, a statistical measurement (e.g., frequency, mean, median, standard deviation, or quantile), or a degree or a relative quantity (e.g., high, medium, and low). A quantitative measure can be a ratio of two quantitative measures. A quantitative measure can be a linear combination of quantitative measures. A quantitative measure may be a normalized measure.

As used herein, “reference sequence” refers to a known sequence used for purposes of comparison with experimentally determined sequences. For example, a known sequence can be an entire genome, a chromosome, or any segment thereof. A reference sequence can align with a single contiguous sequence of a genome or chromosome or chromosome arm or can include non-contiguous segments that align with different regions of a genome or chromosome. Examples of reference sequences include, for example, human genomes, such as, hg19 and hg38.

As used herein, “sample” means anything capable of being analyzed by the methods and/or systems disclosed herein.

As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina, Inc., Pacific Biosciences, Inc., or Applied Biosystems/Thermo Fisher Scientific, among many others.

As used herein, “sequence information” in the context of a nucleic acid polymer means the order and identity of monomer units (e.g., nucleotides, etc.) in that polymer.

As used herein “sequence-variable target region set” refers to a set of target regions that may exhibit changes in sequence such as nucleotide substitutions, insertions, deletions, or gene fusions or transpositions in neoplastic cells (e.g., tumor cells and cancer cells).

As used herein, the terms “somatic mutation” or “somatic variation” are used interchangeably. They refer to a mutation in the genome that occurs after conception. Somatic mutations can occur in any cell of the body except germ cells and accordingly, are not passed on to progeny.

As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject”. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

As used herein, “target-region set” or “set of target regions” or “target regions” or “target regions of interest” or “regions of interest” or “genomic regions of interest” refers to a plurality of genomic loci or a plurality of genomic regions targeted for capture and/or targeted by a set of probes (e.g., through sequence complementarity).

As used herein, “tumor fraction” refers to the proportion of cfDNA molecules that originated from tumor cells for a given sample, or sample-region pair.

As used herein, an “asymmetric adapter” is a double stranded adapter in which the two strands are not completely complementary or are otherwise distinguishable such that synthesis of a complementary sequence of one strand of the adapter results in a sequence that is distinguishable from the sequence of the other strand of the adapter. Examples of asymmetric adapters are Y-shaped adapters and bubble adapters.

As used herein, a “Y-shaped adapter” refers to an adapter comprising two DNA strands comprising complementary and non-complementary parts, wherein the non-complementary parts form single-stranded arms. The adapter can be attached to a sample or insert DNA molecule, e.g., by ligation, such that the complementary (double-stranded) part of the adapter is proximal to the sample or insert DNA molecule. Prior to attachment, the double stranded portion of the Y-shaped adapter may have a blunt end or an overhang, e.g., of one to three nucleotides. The single stranded arms may or may not be of identical length.

As used herein, a “bubble adapter” refers to an adapter comprising two DNA strands comprising a non-complementary part flanked by complementary parts, such that the adapter has a single stranded region located between double-stranded regions. The adapter can be attached to a sample or insert DNA molecule, e.g., by ligation, such that one of the complementary (double-stranded) parts of the adapter is proximal to the sample or insert DNA molecule. Prior to attachment, the double stranded portion of the Y-shaped adapter that would be attached to the insert or sample molecule may have a blunt end or an overhang, e.g., of one to three nucleotides. The single stranded portions of the two strands may or may not be of identical length.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### Samples and Subjects

The disclosure relates to methods of distinguishing artifactual information from real information in workflows involving the sequencing of a DNA sample. In some cases, the DNA sample is obtained or has been obtained from a subject. In some embodiments, the DNA sample may comprise or consist of DNA from a biological sample obtained from a subject. The subject may be a human, a mammal, an animal, a primate, rodent (including mice and rats), or other common laboratory, domestic, companion, service or agricultural animal, for example a rabbit, dog, cat, horse, cow, sheep, goat or pig. Preferably, the DNA sample is from a human. The subject may in some cases have or be suspected of having a cancer, tumor or neoplasm. In other cases the subject may not have cancer or a detectable cancer symptom. The subject may have been treated with one or more cancer therapy, e.g., any one or more of chemotherapies, antibodies, vaccines or biologics. The subject may be in remission, e.g. from a tumor, cancer, or neoplasia (e.g., following treatment such as chemotherapy, surgical resection, radiation, or a combination thereof). The subject may or may not be diagnosed as being susceptible to cancer or any cancer-associated genetic mutations/disorders. In some embodiments, the sample is a DNA sample obtained from a tumor tissue biopsy. The cancer, tumor or neoplasm may generally be of any type, for example a cancer tumor or neoplasm of the lung, colon, rectum (or colorectum), kidney, breast, prostate, or liver, or other type of cancer as described herein. In some embodiments, the sample is obtained from a subject in remission from a tumor, cancer, or neoplasia (e.g., following chemotherapy, surgical resection, radiation, or a combination thereof). In any of the foregoing embodiments, the pre-cancer, cancer, tumor, or neoplasia or suspected pre-cancer, cancer, tumor, or neoplasia may be of the bladder, head and neck, lung, colon, rectum, kidney, breast, prostate, skin, or liver. In some embodiments, the pre-cancer, cancer, tumor, or neoplasia or suspected pre-cancer, cancer, tumor, or neoplasia is of the lung. In some embodiments, the pre-cancer, cancer, tumor, or neoplasia or suspected pre-cancer, cancer, tumor, or neoplasia is of the colon or rectum. In some embodiments, the pre-cancer, cancer, tumor, or neoplasia or suspected pre-cancer, cancer, tumor, or neoplasia is of the breast. In some embodiments, the pre-cancer, cancer, tumor, or neoplasia or suspected pre-cancer, cancer, tumor, or neoplasia is of the prostate. In any of the foregoing embodiments, the subject may be a human subject. In some embodiments, the sample is obtained from a subject having a stage I cancer, stage II cancer, stage III cancer or stage IV cancer.

In some embodiments, the subject may have an infection, a transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed as being susceptible to cancer or any cancer-associated genetic mutations/disorders.

The biological sample can be any biological sample isolated from a subject. Biological samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. In some embodiments, biological samples are body fluids, particularly blood and fractions thereof, or urine. A sample can be in the form originally isolated from a subject or can have been subjected to further processing to remove or add components, such as cells, or enrich for one component relative to another. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis.

In a preferred embodiment, the DNA sample comprises cell-free DNA. In another preferred embodiment the DNA sample is a DNA sample from a formalin fixed paraffin embedded (FFPE) sample.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

In some embodiments, the DNA sample is from plasma. The volume of plasma used to obtain the DNA sample can depend on the desired read depth for sequenced regions. Exemplary volumes are 0.4-40 ml, 5-20 ml, 10-20 ml. For example, the volume can be 0.5 mL, 1 mL, 5 mL 10 mL, 20 mL, 30 mL, or 40 mL. A volume of sampled plasma may be 5 to 20 mL.

A sample can comprise various amounts of DNA that contains genome equivalents. For example, a sample of about 30 ng DNA can contain about 10,000 (104) haploid human genome equivalents and, in the case of cell free DNA (cfDNA), about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., nucleic acids from cells and cell-free nucleic acids of the same subject, and nucleic acids from cells and cell-free nucleic acids of different subjects. In some embodiments, the nucleic acid may be DNA. A sample can comprise DNA carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant, wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

The DNA sample may be or comprise cell free nucleic acids or cfDNA. The cfDNA may be obtained from a test subject, for example as described above. For example, the sample for analysis may be plasma or serum containing cell-free nucleic acids. “Cell-free DNA” “cfDNA molecules,” or “cfDNA”, for example, include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) in vivo into a fluid found in the organism, and may be obtained by obtaining a sample of the fluid without the need to perform an in vitro cell lysis step. In other words, cell-free nucleic acids or cfDNA are nucleic acids or DNA not contained within or otherwise bound to a cell, or the nucleic acids or DNA remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA). In some embodiments, cell free nucleic acids are produced by tumor cells. In some embodiments, cell free nucleic acids are produced by a mixture of tumor cells and non-tumor cells.

Exemplary amounts of cell-free nucleic acids (e.g. cfDNA) in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng cell-free nucleic acid molecules from samples.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, DNA or protein for sequencing (e.g., bisulfite sequencing), hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

The methods disclosed herein are also particularly suited for the analysis of DNA from formalin-fixed paraffin-embedded (FFPE) tissue samples. While the formalin fixation process adequately preserves the ultrastructure of the tissues, it results in various types of damage to the DNA within the tissues, such as nicks in the DNA. As explained elsewhere herein, these nicks can lead to synthesis of regions of the DNA molecule in the end repair process. The methods disclosed herein allow for these regions to be identified and the sequence data to be interpreted accordingly.

### End Repair and A-Tailing

End repair refers to methods for repairing DNA by the conversion of non-blunt ended DNA into blunt ended DNA. Sequencing workflows typically use end repair to make ends of DNA molecules compatible with adapters, which are subsequently ligated onto the DNA. Fragmented and/or damaged DNA (e.g. cfDNA or DNA from FFPE samples) often contain non-blunt ends, which contain 3′overhangs and/or 5′overhangs. A 3′overhang refers to the 3′ end of a DNA strand which extends beyond the 5′end of the paired strand, resulting in one or more unpaired nucleotides at the 3′end of the DNA strand. Conversely, a 5′overhang refers to the 5′ end of a DNA strand which extends beyond the 3′end of the paired strand, resulting in one or more unpaired nucleotides at the 5′end of the DNA strand.

The process of end repair involves the conversion of double-stranded DNA with 3′overhangs and/or 5′overhangs to double-stranded DNA without overhangs. This can be done using an enzymes such as T4 DNA polymerase and/or Klenow fragment. The 3′ to 5′ exonuclease activity of these enzymes removes the 3′ends at 3′overhangs and the 5′ to 3′ polymerase activity of these enzymes extends the 3′ ends at 5′ overhangs to remove the 5′ overhang, thereby generating a blunt-ended DNA molecule. In order to fill in these 5′ overhangs, end repair is conducted in the presence of dATP, dCTP, dGTP and dTTP. End repair can also include a second step, which involves the addition of a phosphate group to the 5′ ends of DNA, by an enzyme such as polynucleotide kinase. This makes the 5′ends of the end-repaired DNA molecules compatible with the subsequent action of DNA polymerases and DNA ligases.

As used herein, the term “A-tailing” refers to the addition of a single deoxyadenosine residue to the end of a blunt-ended double-stranded DNA fragment to form a 3′ deoxyadenosine single-base overhang. Such A tailing reactions are conducted with polymerases which have the ability to add a non-templated A to the 3′ end of a blunt, double-stranded DNA molecule. Polymerases capable of A-tailing typically do not possess 3′-5′ exonuclease activity. When A-tailing is performed as a separate reaction to end repair, it is typically conducted in the presence of dATP, but the absence of dCTP, dTTP and dGTP. A-tailed fragments are not compatible for self-ligation (i.e., self-circularizatian and concantenation of the DNA), but they are compatible with 3′ T-overhangs, which can be used on adapters. Methods comprising end repair, A-tailing and ligation to adapters with 3′ T-overhangs can result in higher efficiency ligation, compared to blunt ended ligation, as blunt ligation can lead to self-ligation of the adapters and/or DNA molecules.

In some cases, the methods disclosed herein comprise end repair of the DNA molecules followed by blunt end ligation of adapters. In other cases, the methods disclosed herein comprise end repair of the DNA molecules followed by A-tailing and sticky-end ligation of T-tailed adapters. When the methods disclosed herein comprise an A-tailing step, it may be performed separately from the end repair with an intervening reaction clean-up step or it may be performed in the same reaction as the end repair (e.g. using NEBNext® Ultra™ II End Repair/dA-Tailing Module (E7546)). In instances wherein the A-tailing reaction is performed in the same reaction as end repair, the sticky-end ligation may be performed with a mixture of T-tailed adapters and C-tailed adapters.

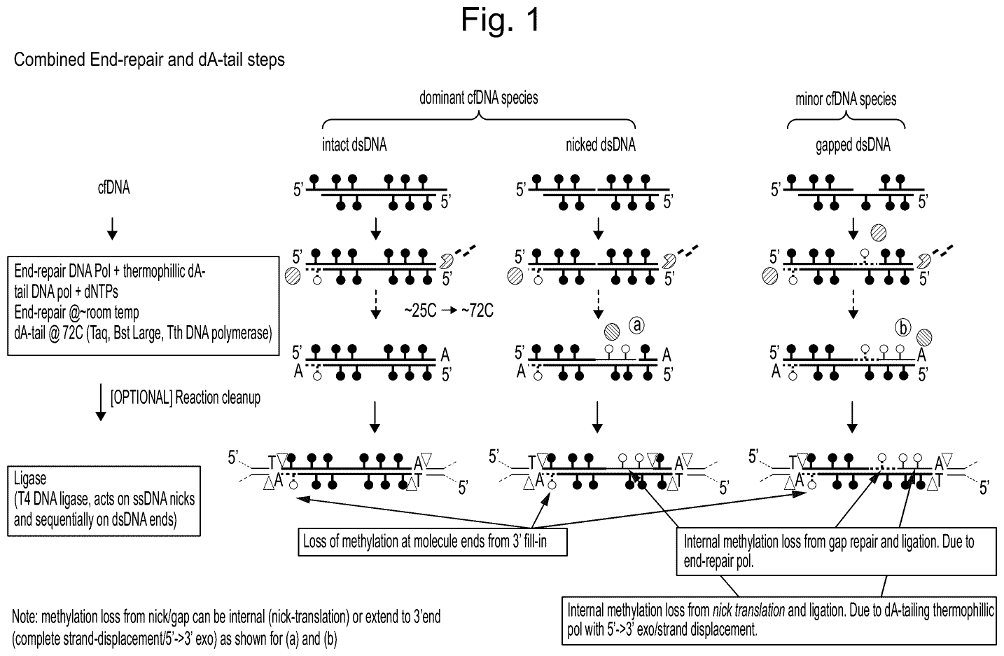

The inventors have investigated the effect of end repair and A-tailing steps on the DNA molecules, and its impact on corresponding sequence data. As shown in FIGS. 1-10 (discussed in detail below), end repair and A tailing reactions can have varying impacts on the composition of the DNA molecule, dependent on the exact workflow and reaction components used. These reactions can lead to the synthesis of regions at the 3′ends of DNA strands, but also the synthesis of internal regions through nick translation and through gap filling followed by ligation. The impact of such synthesis is shown in FIGS. 1-10 in the context of its effect on cytosine methylation.

FIG. 1 shows a schematic of the impact of combined end repair and A tailing steps on intact double-stranded DNA, nicked double-stranded DNA and gapped double-stranded DNA. In all types of DNA molecule shown, end repair can lead to 3′fill in with unmethylated cytosines, which may not reflect the true methylation status of that position in the DNA molecule prior to the generation of the 5′overhang. Inferring the methylation status from this end repaired DNA molecule may lead to incorrect methylation data at this position. In nicked DNA, polymerases which contain 5′ to 3′ exonuclease activity and/or strand displacement activity can lead to the synthesis of internal regions of the DNA molecule through nick translation. If the end repair reaction is conducted with non-methylated deoxycytidine triphosphate (dCTP), the synthesized regions will incorporate the non-methylated dCTP, potentially at positions which initially comprised methylated cytosines. Using sequence data from these synthesized regions to infer the methylation status of the original DNA molecule may therefore lead to inaccurate data. In gapped DNA, both the DNA polymerases used in end repair and A tailing can lead to the generation of synthesized regions. The gaps can be filled in with DNA polymerases used in the end repair reaction, regardless of whether they possess 5′ to 3′ exonuclease activity or strand displacement activity. After this gap filling, a nick will still exist between the synthesized region and the region of the original DNA molecule 3′ of the gap. The A-tailing enzymes may then introduce further synthesized regions through nick translation, as described for the nicked DNA. This synthesized region may extend to the 3′end of the DNA molecule. As before, using these synthesized regions to infer the methylation status of the original DNA molecules may lead to incorrect results.

In some embodiments, the end-repair and the A-tailing reactions are performed in a single tube. In such cases, the A tailing reaction can be performed at a higher temperature than the end repair. Optionally, end repair is performed at ambient temperature (e.g. 15-35° C.) and A tailing is performed at a temperature over 60° C. The A tailing reaction can be performed using a thermostabile polymerase (e.g. Taq DNA polymerase, Tfl DNA polymerase, Bst DNA Polymerase, Large Fragment or Tth DNA polymerase) and the method further comprises increasing temperature of the sample after the end repair to inactivate the polymerase used in end repair (e.g. T4 DNA polymerase or Klenow fragment). In some embodiments the A-tailing is performed using a DNA polymerase that: (i) does not possess 5′-3′ exonuclease activity; and/or (ii) is not a strand displacing DNA polymerase. These properties reduce the ability of the DNA polymerase to extend from nick. This reduces the level of synthesis which may occur during the end repair and A-tailing reactions thus reducing the proportion of sequencing data that may be filtered out as potentially containing artifactual data. Accordingly, in some embodiments, the A-tailing is performed using a DNA polymerase that cannot extend from a nick in the DNA such as HemoKlen Taq. In other embodiments, the A-tailing is performed using Taq DNA polymerase. In other embodiments, the A-tailing is performed using Tfl polymerase, Bst DNA Polymerase, Large Fragment or Tth polymerase.

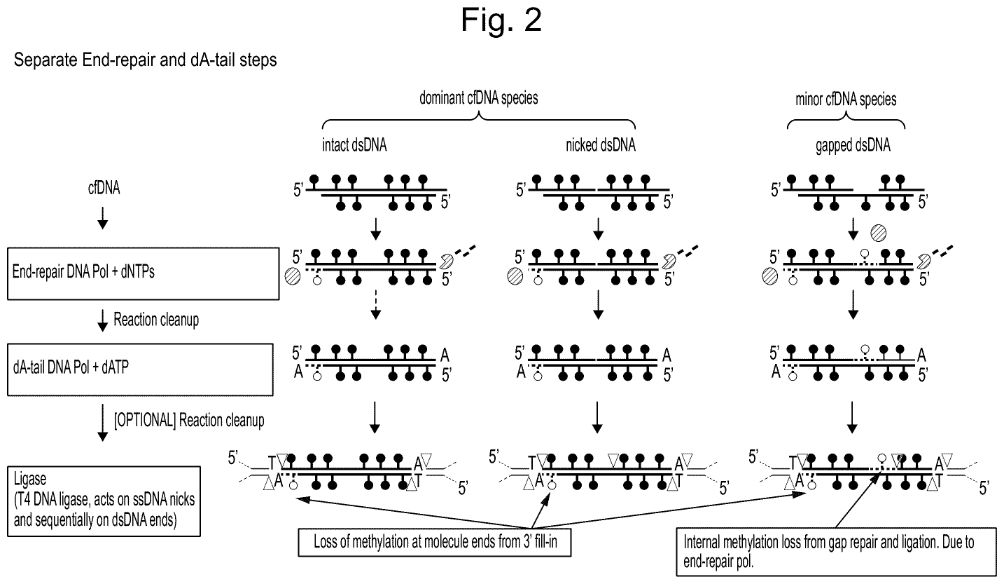

FIG. 2 shows a schematic of the potential impact of separate end repair and A tailing steps on intact double-stranded DNA, nicked double-stranded DNA and gapped double-stranded DNA. In order to reduce the level of synthesized regions, the end repair reaction can be performed using DNA polymerases can be used which lack 5′ to 3′ exonuclease activity and/or strand displacement activity (e.g. T4 DNA polymerase or Klenow fragment). This approach means that nick translation is reduced such that a larger portion of the sequencing data can be used in the disclosed methods for methylation analysis, because less sequencing data will derive from synthesized regions.

In all types of DNA molecule shown in FIG. 2, end repair can lead to 3′fill in with unmethylated cytosines, which may not reflect the true methylation status of that position in the DNA molecule prior to the generation of the 5′overhang. Inferring the methylation status from this end repaired DNA molecule may lead to incorrect data at this position. In nicked DNA and gapped DNA, nick translation is reduced in end repair through the use of polymerases which lack 5′ to 3′ exonuclease activity and/or strand displacement activity. The separation of the end repair and A tailing reaction by a reaction clean-up means that only dATP (not dCTP, dTTP or dGTP) is present in the A tailing reaction. This means that efficient nick translation cannot occur in the A tailing reaction because the three of the four nucleotide components are not present in the reaction mixture. In gapped DNA, the gaps can be filled in with DNA polymerases used in the end repair reaction, regardless of whether they possess 5′ to 3′ exonuclease activity or strand displacement activity. These filled gaps thereby generate synthesized regions. As before, using these synthesized regions to infer the methylation status of the original DNA molecules may lead to incorrect results. The extent of synthesized regions in such a workflow, however, is greatly reduced relative to workflows wherein the end repair and A-tailing reactions are performed in a single reaction, as shown in FIG. 1.

As such, in preferred embodiments, the methods disclosed herein the end repair is performed with a polymerase which lacks 5′ to 3′ exonuclease activity and/or strand displacement activity. In some cases, the polymerase used in the end repair reaction may be Q5® High-Fidelity DNA Polymerase, Q5U® Hot Start High-Fidelity DNA Polymerase, Phusion® High-Fidelity DNA Polymerase, Hemo KlenTaq, phi29 DNA Polymerase, T7 DNA Polymerase, DNA Polymerase I (E. coli), DNA Polymerase I, Large (Klenow) Fragment (“Klenow fragment”) or T4 DNA Polymerase. In some embodiments, the polymerase used in the end repair is T4 DNA Polymerase or Klenow fragment.

In some embodiments, the methods disclosed herein comprise an A tailing reaction after the end repair and before the ligation reaction, wherein the end repair and A tailing reactions are separated by a reaction cleanup. The A tailing reaction is typically performed in the presence of dATP, but in the absence of dCTP, dTTP and dGTP. In some embodiments, the A tailing reaction is performed using Klenow Fragment lacking 3′-5′ exonuclease activity.

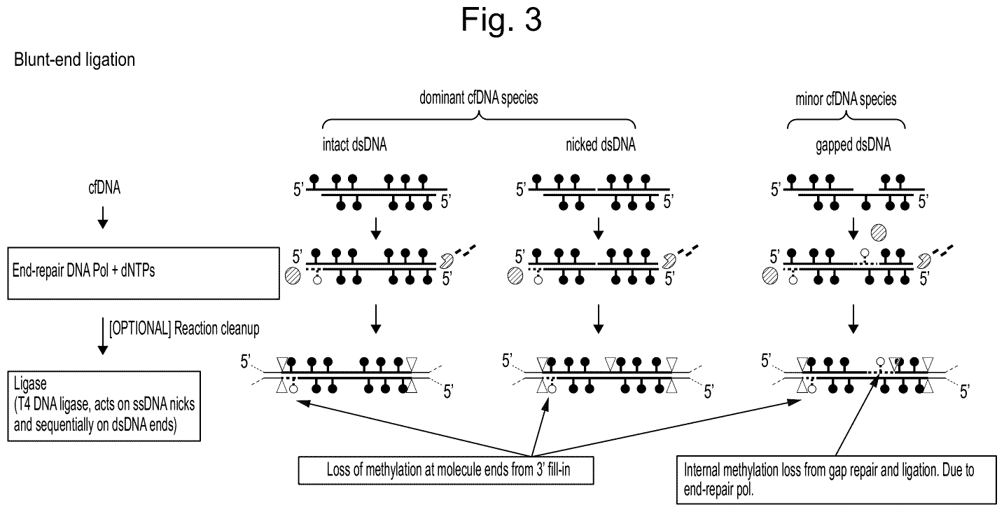

FIG. 3 shows a schematic of the potential impact of end repair and blunt end ligation on intact double-stranded DNA, nicked double-stranded DNA and gapped double-stranded DNA. In order to reduce the level of synthesized regions, the end repair reaction can be performed using DNA polymerases can be used which lack 5′ to 3′ exonuclease activity and/or strand displacement activity (e.g. T4 DNA polymerase or Klenow fragment). This approach means that nick translation is reduced such that a larger portion of the sequencing data can be used in the disclosed methods for methylation analysis, because less sequencing data will derive from synthesized regions.

In all types of DNA molecule shown in FIG. 3, end repair can lead to 3′fill in with unmethylated cytosines, which may not reflect the true methylation status of that position in the DNA molecule prior to the generation of the 5′overhang. Inferring the methylation status from this end repaired DNA molecule may lead to incorrect data at this position. In nicked DNA and gapped DNA, nick translation is reduced in end repair through the use of polymerases which lack 5′ to 3′ exonuclease activity and/or strand displacement activity. In gapped DNA, the gaps can be filled in with DNA polymerases used in the end repair reaction, regardless of whether they possess 5′ to 3′ exonuclease activity or strand displacement activity. These filled gaps thereby generate synthesized regions. As before, using these synthesized regions to infer the methylation status of the original DNA molecules may lead to incorrect results. The extent of synthesized regions in such a workflow, however, is greatly reduced relative to workflows wherein end repair and A-tailing reactions are performed in a single reaction, as shown in FIG. 1.

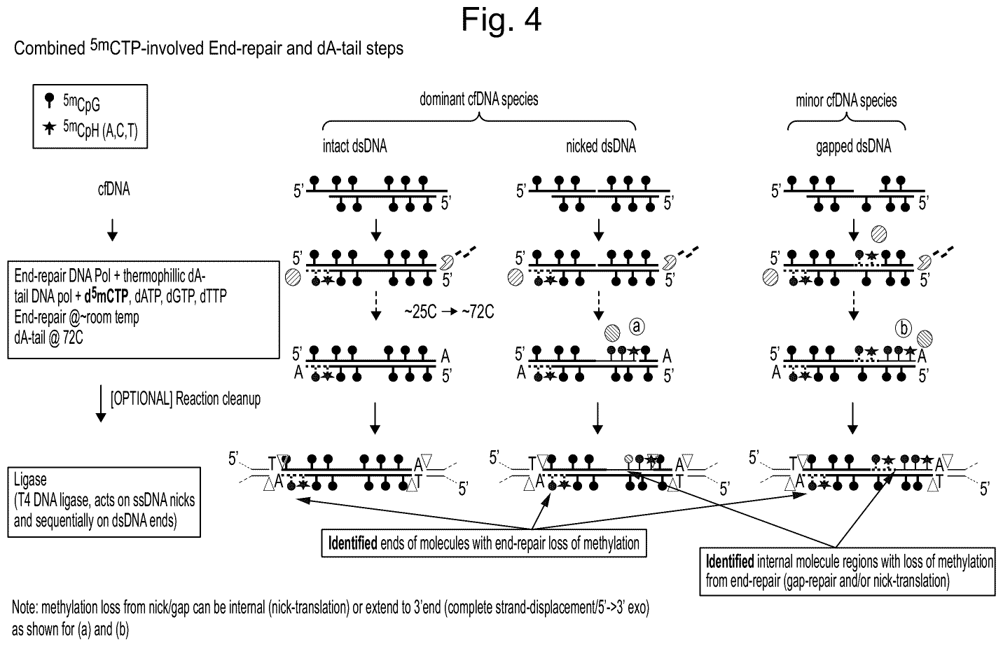

FIG. 4 is a repeat of the schematic shown in FIG. 1, but with the use of a dNTP comprising a modified base in the combined end repair and A-tailing reaction, which, in this example is methylated cytosine (5mC). It shows that the methylated cytosines are incorporated in the synthesized regions at both CpG sites and CpH sites (i.e. CpA, CpC and CpT sites). While methylation of cytosines in non-CpG contexts has been described, it is thought to comprise 0.02% of total methyl-cytosine in differentiated somatic cells (Jang et al. Genes (Basel). 2017 June; 8(6): 148). The presence of methylated cytosine in non-CpG contexts in the end-repaired DNA can therefore be interpreted as being introduced during the end repair and/or A-tailing reactions. Using a dNTP comprising a modified base can therefore be used to effectively label the synthesized regions of the end repaired DNA.

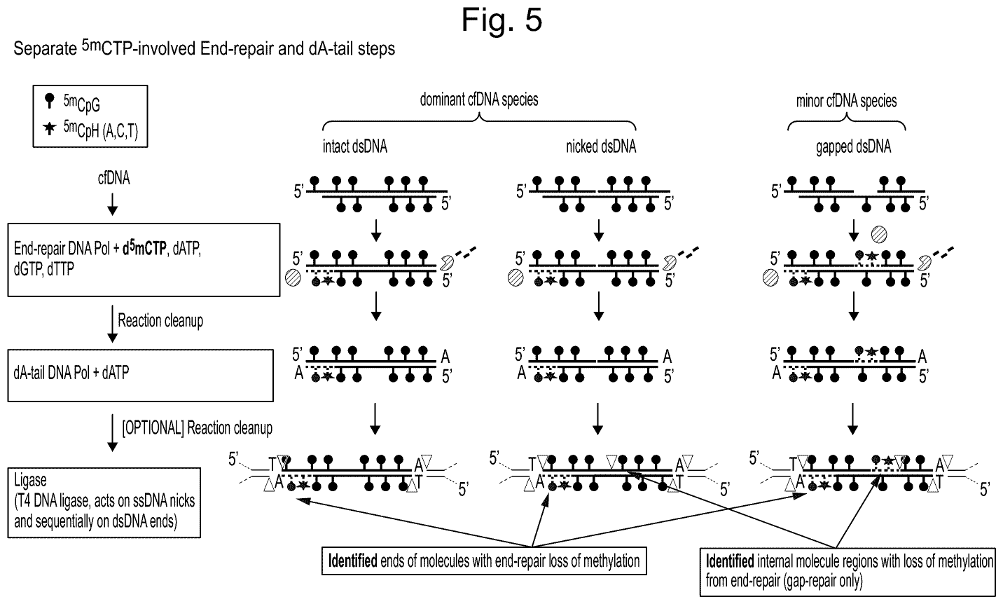

FIG. 5 is a repeat of the schematic shown in FIG. 2, but with the use of a dNTP comprising a modified base in the end repair, which, in this example is methylated cytosine (5mC). As with FIG. 4, it shows that the methylated cytosines are incorporated in the synthesized regions at both CpG sites and CpH sites (i.e. CpA, CpC and CpT sites). Using a dNTP comprising a modified base can therefore be used to effectively label the synthesized regions of the end repaired DNA.

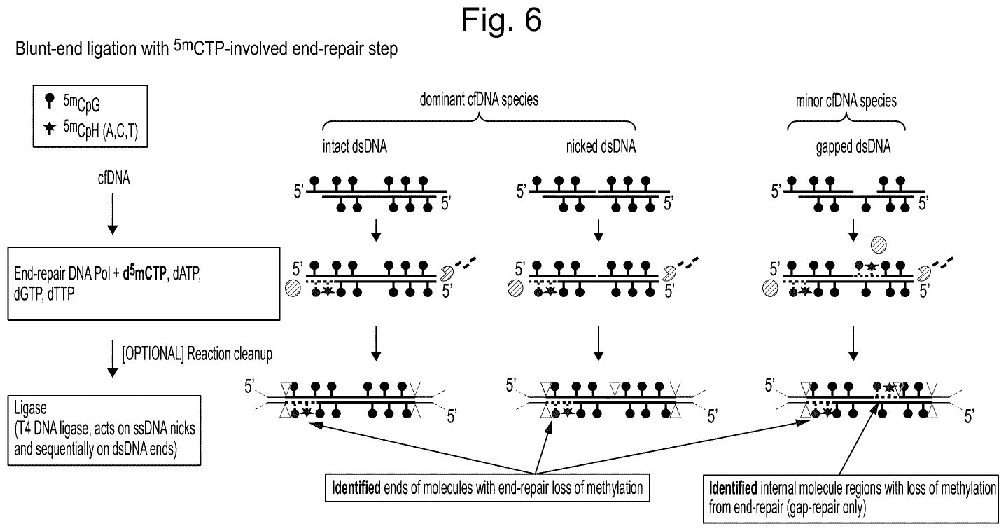

FIG. 6 is a repeat of the schematic shown in FIG. 3, but with the use of a dNTP comprising a modified base in the end repair, which, in this example is methylated cytosine (5mC). As with FIG. 4, it shows that the methylated cytosines are incorporated in the synthesized regions at both CpG sites and CpH sites (i.e. CpA, CpC and CpT sites). Using a dNTP comprising a modified base can therefore be used to effectively label the synthesized regions of the end repaired DNA.

The dNTP that comprises a modified base may comprise any modified base wherein the presence or the absence of the modification can be detected by a type of modification sensitive sequencing. The modified base may be 4-methylcytosine (4mC), 5-methylcytosine (5mC), 5-hydroxymethyl-cytosine (5hmC), N6-methyladenosine (6 mA), bromodeoxyuridine (BrdU), 5-fluorodeoxyuridine (FldU), 5-iododeoxyuridine (IdU), 5-ethynyldeoxyuridine (EdU) and/or 8-oxoguanine (8oxoG).

When a dNTP comprising a modified base is used, it may be used in place of the equivalent unmodified base in the end repair reaction. For instance, if a dCTP comprising 5mC is used in the end repair reaction, there may be no dCTP comprising an unmodified cytosine. This would ensure that dCTPs incorporated into the DNA molecule during the end repair reaction contain 5mC. In some embodiments, multiple types of dNTP comprising a modified base are used in the end repair. For example, dATP comprising 6 mA and dCTP comprising 5mC can be used in the end repair reaction in place of dATP comprising unmodified adenine and dCTP comprising unmodified cytosine. The use of multiple types of dNTP comprising a modified base is advantageous because it provides increased resolution in defining the regions of the end-repaired DNA molecule which have been synthesized during the end repair reaction. This is because, in this example, the end of a synthesized region can be defined as the first unmodified adenine or unmodified cytosine after a stretch of containing 6mAs and/or 5mCs, rather than relying on the detection of solely an unmodified adenine or solely an unmodified cytosine.

The modification sensitive sequencing method used will depend on the type of modified base used in the end-repair reaction such that the specific modification can be detected. Exemplary conversion-based methods are described above alongside the base modification which they can detect. Moreover, nanopore-based sequencing can be used to detect 4mC, 5mC, 5hmC, 6 mA, BrdU, FdU, IdU, and EdU, and single-molecule real time (SMRT) sequencing from Pacific Biosciences can be used to detect 4mC, 5mC, 5hmC, 6 mA, and 8oxoG.

### Ligation to Adapters

Once the DNA has been end-repaired it can subjected to blunt-end ligation with blunt-ended adapters, in cases where A-tailing is not performed, or sticky end ligation with T-tailed adapters, when A tailing is performed. DNA molecules can be ligated to adapters at either one end or both ends. DNA molecules can be ligated with at least partially double stranded adapter (e.g., a Y shaped or bell-shaped adapter). In embodiments wherein the modification-sensitive sequencing comprises a conversion procedure, the ligation step can take place before or after the conversion step. In some embodiments, the ligation step is performed after the conversion step.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample DNA molecule. In some instances, two adapters can be ligated to a single sample DNA molecule, with one adapter ligated to each end of the sample nucleic acid molecule.

In some embodiments, the ligase used in ligation reactions can act on both single strand DNA nicks and double stranded DNA ends. In some cases, the ligase is T4 DNA ligase or T3 DNA ligase. Adapters can include nucleic acid primer binding sites to permit amplification of a sample DNA molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given DNA molecule. Adapters of the same or different sequence can be linked to the respective ends of a sample DNA molecule. In some cases, adapters of the same or different sequence are linked to the respective ends of the DNA molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a DNA molecule to be analyzed. Other exemplary adapters include T-tailed, C-tailed or hairpin shaped adapters. For example, a hairpin shaped adaptor can comprise a complementary double stranded portion and a loop portion, where the double stranded portion can be attached (e.g. ligated) to a double-stranded polynucleotide. Hairpin shaped sequencing adaptors can be attached to both ends of a polynucleotide fragment to generate a circular molecule, which can be sequenced multiple times. The adapters used in the methods of the present disclosure comprise one or more known modified nucleosides, such as methylated nucleosides. In instances where two adapters are ligated to a sample nucleic acid (one at each end), either or both of the adapters may comprise one or more known modified nucleosides. Typically, the primer binding site(s), sequencing primer binding site(s), sample index(es) and/or molecular barcode(s), if present, do not comprise the known modified nucleosides that change base pairing specificity as a result of the conversion procedure.

In some embodiments, adapters may be added to the DNA or a subsample thereof. Adapters can be ligated to DNA at any point in the methods herein. In some embodiments, adapters are ligated to the DNA of a sample or subsample thereof prior to annealing primers to the DNA for capture probe generation. In some such embodiments, the adapter-ligated DNA is amplified prior to annealing primers to the DNA for capture probe generation. In some embodiments, adapters are ligated to the DNA of a sample or subsample thereof before the DNA is contacted with the capture probes. In some embodiments, the DNA to which the adapters are ligated is in the same sample or subsample as the DNA used as a template to generate capture probes. In some embodiments, the DNA to which the adapters are ligated is in a different sample or subsample, e.g., a second sample or a second subsample of a first sample, than the DNA used as a template to generate capture probes. In some embodiments, the adapters ligated to DNA captured by the capture probes.

In some embodiments, the primers used to generate capture probes are not complementary to adapters, and the resulting capture probes therefore do not comprise adapters. Adapter-ligated DNA can therefore be selectively amplified in the presence of capture probes that do not comprise adapters. Similarly, adapter-ligated DNA can be separated from DNA that does not comprise adapters.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample or portion of the DNA is partitioned, comprising contacting the DNA with an agent that preferentially binds to nucleic acids bearing an epigenetic modification. The nucleic acids are partitioned into at least two partitioned subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

### Molecular Tagging

In some embodiments, the DNA molecules of the sample may be tagged with sample indexes and/or molecular barcodes (referred to generally as “tags”).

Tags can be molecules, such as nucleic acids, containing information that indicates a feature of the molecule with which the tag is associated. For example, DNA molecules can bear a sample tag or sample index (which distinguishes molecules in one sample from those in a different sample), a partition tag (which distinguishes molecules in one partition from those in a different partition) and/or a molecular tag/molecular barcode (which distinguishes different molecules from one another (in both unique and non-unique tagging scenarios)).

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member.

Tags can be used to label the individual polynucleotide population partitions so as to correlate the tag (or tags) with a specific partition. Alternatively, tags can be used in embodiments of the disclosure that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g. by blunt-end ligation or sticky-end ligation), or overlap extension polymerase chain reaction (PCR), among other methods. Such adapters are ultimately joined to the sample DNA molecule. In other embodiments, one or more rounds of amplification cycles (e.g., PCR amplification) may be applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after any conversion procedure. In the case of molecular barcodes and/or sample indexes being introduced through amplification processes, the conversion step will occur before the molecular barcodes and/or sample indexes are introduced. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample DNA molecule. In some embodiments, tags are predetermined or random or semi-random sequence oligonucleotides. In some embodiments, the tag(s) may together be less than about 500, 200, 100, 50, 20, 10, 9, 8, 7, 6, 5, 4, 3, 2, or 1 nucleotides in length. Typically tags are about 5 to 20 or 6 to 15 nucleotides in length. The tags may be linked to sample DNA molecules randomly or non-randomly.

In some embodiments, each sample or partition (discussed below) is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation as part of an adapter) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original DNA molecule in the sample, start and stop genomic positions corresponding to the sequence of the original DNA molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original DNA molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene).

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes. For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths). Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments a format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, the method includes adding one or more internal control DNAs and forward and reverse primers for amplifying the internal control DNAs. The internal control DNAs may be added before amplification using the primers that anneal upstream and downstream of the rearrangement breakpoints. The forward and reverse primers for amplifying the internal control DNAs may be included with, or added at the same time as, the primers that anneal upstream and downstream of the rearrangement breakpoints. The internal control DNAs may comprise or consist of sequences that do not occur in the genome of the subject, or that do not occur in the genome of the species of which the subject is a member (e.g., the human genome). The forward and/or reverse primers for amplifying the internal control DNAs may comprise sequences that are not complementary to any sequence in the genome of the subject, e.g., the human genome. The internal control DNAs may be used to ensure that the amplification process proceeded as designed. As such, the method may comprise detecting (e.g., sequencing) molecules amplified from and/or captured by the one or more internal control DNAs. The method can comprise comparing an amount of internal control DNAs (e.g., number of molecules or reads detected that correspond to an internal control DNA sequence) to a predetermined threshold, and either rejecting sequencing results if the predetermined threshold is not met or accepting sequencing results if the predetermined threshold is met. The predetermined threshold may be established, e.g., based on historical data or by testing the method on samples of DNA from test subjects, such as healthy volunteers. For example, amplification and detection of the one or more internal control DNAs provides confirmation that the amplification process proceeded properly, thus reducing the likelihood of a false negative.

### Modification Sensitive Sequencing

The methods disclosed herein use modification sensitive sequencing to detect the modification status of the at least one type of dNTP comprising a modified base used in the end repair reaction. “Modification sensitive sequencing” refers to any sequencing workflow which is capable of distinguishing at least two modification states of a nucleotide bases. These two states may be: (i) whether a base is modified or not (e.g. 5mC and/or 5hmC vs unmethylated cytosine); or (ii) the type of modification which a base exhibits (e.g. 5mC vs 5hmC). Modification sensitive sequencing does not necessarily require that a specific type of modification is identified as present or absent at a specific position, just whether one or more modification types (e.g. 5mC and 5hmC) is present or absent. For instance, in some embodiments, modification sensitive sequencing includes sequencing comprising a bisulfite conversion step which can distinguish 5mC and 5hmC from unmethylated C, but it cannot distinguish between 5mC and 5hmC.

The type of modification sensitive sequencing used will, of course, depend on the type of modified base(s) used in the end repair, such that the type of modification sensitive sequencing will be able to detect at least the presence or absence of at least that modified base. Table 1 summarizes exemplary forms of modification sensitive sequencing with the type of modified bases detectable with these methods. These are described in more detail below.

TABLE 1
Exemplary modification sensitive sequencing methods

| Sequencing | Base modification |
|---|---|
| method | detectable |
| Bisulfite | 5mC and 5hmC |
| conversion | distinguishable |
| | from C |
| Oxidative bisulfite | 5mC distinguishable |
| conversion | from 5hmC and C |
| Tet-assisted bisulfite | 5hmC distinguishable |
| (TAB) conversion | from 5mC and C |
| APOBEC-coupled epigenetic | 5hmC distinguishable |
| (ACE) conversion | from 5mC and C |
| Enzymatic methyl-seq | 5mC and 5hmC |
| (EM-seq). | distinguishable |
| | from C |
| Single-enzyme | 5mC and 5hmC |
| 5-methylctyosine | distinguishable |
| sequencing (SEM-seq) | from C |
| Tet-assisted pyridine | 5mC and 5hmC |
| borane sequencing | distinguishable |
| (TAPS) | from C |
| Nanopore-based | 4mC, 5mC, 5hmC, 6mA, BrdU, |
| sequencing | FldU, IdU and EdU. |
| Single-molecule real- | 4mC, 5mC, 5hmC, |
| time sequencing | 6mA, and 8oxoG. |
| (SMRT) | |

As outlined below, there are various methods of detecting and/or identifying modified nucleosides that rely on a conversion procedure that changes the base-pairing specificity of a nucleoside, based on the modification status of the nucleosides. These changes of base-pairing specificity can then be detected, and thus the modification status of the nucleoside inferred, by sequencing. Together, the conversion procedure and the sequencing itself constitutes one form of modification aware sequencing, as referred to herein.

In some cases, the conversion procedure used in the methods of the disclosure is one that changes the base pairing specificity of a modified nucleoside (e.g. methylated cytosine), but does not change the base pairing specificity of the corresponding unmodified nucleoside (e.g. cytosine) or does not change the base pairing specificity of any un-modified nucleoside (e.g. cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as TAPS may in some cases be preferred over methods such as bisulfite sequencing and EM-seq because they are less destructive (especially important for low yield samples such as cfDNA or FFPE samples) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized regions of interest, these non-random (localized) non-conversion events can appear as false negatives (non-methylated regions). Random non-conversion methods can maximally affect a low percent of bases within a region, and thus the specificity of methylation change detection can be maximized (reduce false positives) by placing a threshold on percentage of bases within a region that are methylated/non-methylated. Hence, in some cases, a conversion procedure that does not involve denaturation is preferred.

In other cases, the conversion procedure used in the methods of the disclosure is one that changes the base pairing specificity of an unmodified nucleoside (e.g. cytosine), but does not change the base pairing specificity of the corresponding modified nucleoside (e.g. methylated cytosine such as 5hmC and/or 5mC). Such methods include, for example, bisulfite sequencing.

The skilled person can select a suitable method according to their needs, including which nucleoside modifications are to be detected and/or identified and which type of modified base is used in the end repair reaction.

In some embodiments, the conversion procedure converts modified nucleosides. In some embodiments, the conversion procedure which converts modified nucleosides comprises Tet-assisted conversion with a substituted borane reducing agent, optionally wherein the substituted borane reducing agent is 2-picoline borane, borane pyridine, tert-butylamine borane, ammonia borane or pyridine borane. In Tet-assisted pic-borane conversion with a substituted borane reducing agent conversion, a TET protein is used to convert 5mC and 5hmC to 5caC, without affecting unmodified C. 5caC, and 5fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). DHU is read as a T in sequencing. Sequencing of the converted DNA identifies positions that are read as cytosine as being unmodified C positions. Meanwhile, positions that are read as T are identified as being T, 5mC, 5fC, 5caC, or 5hmC. Performing TAP conversion thus facilitates identifying positions containing unmodified C using the sequence reads obtained.

Hence, in these embodiments, the end repair reaction can be performed with dNTPs, wherein the at least one type of dNTP comprises a 5mC or 5hmC, and regions synthesized during the end repair reaction can be identified as those regions comprising 5mC or 5hmC (via T being called at positions which are C in the reference) at non-CpG positions. This procedure encompasses Tet-assisted pyridine borane sequencing (TAPS), described in further detail in Liu et al. 2019, supra. In this method Tet enzyme is used to progressively oxidize 5mC and 5hmC to 5fC or 5caC, then pyridine borane deaminates 5fC, 5CaC to DHU, amplified as T.

Alternatively, protection of 5hmC (e.g., using PGT) can be combined with Tet-assisted conversion with a substituted borane reducing agent, e.g. as described above. In this method (TAPS-0), 5hmC can be protected from conversion, for example through glucosylation using β-glucosyl transferase (OGT), forming (forming 5-glucosylhydroxymethylcytosine) 5ghmC. This is described in Yu et al., Cell 2012; 149: 1368-80. Treatment with a TET protein such as mTet1 then converts 5mC to 5caC but does not convert C or 5ghmC. 5caC is then converted to DHU by treatment with pic-borane or another substituted borane reducing agent such as borane pyridine, tert-butylamine borane, or ammonia borane, also without affecting unmodified C or 5ghmC. Sequencing of the converted DNA identifies positions that are read as cytosine as being either 5hmC or unmodified C positions. Meanwhile, positions that are read as T are identified as being T, 5fC, 5caC, or 5mC. Performing TAPSβ conversion on a sample as described herein thus facilitates distinguishing positions containing unmodified C or 5hmC on the one hand from positions containing 5mC using the sequence reads obtained. Hence, in these embodiments, the end repair reaction can be performed with dNTPs, wherein the at least one type of dNTP comprises a 5mC, and regions synthesized during the end repair reaction can be identified as those regions comprising 5mC (via T being called at positions which are C in the reference) at non-CpG positions. For an exemplary description of this type of conversion, see, e.g., Liu et al., Nature Biotechnology 2019; 37:424-429.

In some embodiments, the conversion procedure converts modified nucleosides. In some embodiments, the conversion procedure which converts modified nucleosides comprises chemical-assisted conversion with a substituted borane reducing agent, optionally wherein the substituted borane reducing agent is 2-picoline borane, borane pyridine, tert-butylamine borane, borane pyridine or ammonia borane. In chemical-assisted conversion with a substituted borane reducing agent, an oxidizing agent such as potassium perruthenate (KRuO₄) (also suitable for use in ox-BS conversion) is used to specifically oxidize 5hmC to 5fC. Treatment with pic-borane or another substituted borane reducing agent such as borane pyridine, tert-butylamine borane, or ammonia borane converts 5fC and 5caC to DHU but does not affect 5mC or unmodified C. Sequencing of the converted DNA identifies positions that are read as cytosine as being either 5mC or unmodified C positions. Meanwhile, positions that are read as T are identified as being T, 5fC, 5caC, or 5hmC. Performing this type of conversion as described herein thus facilitates distinguishing positions containing unmodified C or 5mC on the one hand from positions containing 5hmC using the sequence reads obtained. Hence, in these embodiments, the end repair reaction can be performed with dNTPs, wherein at least one type of dNTP comprises a 5hmC, and regions synthesized during the end repair reaction can be identified as those regions comprising 5hmC (via T being called at positions which are C in the reference) at non-CpG positions. For an exemplary description of this type of conversion, see, e.g., Liu et al., Nature Biotechnology 2019; 37:424-429.

Exemplary conversion procedures that change the base-pairing specificity of modified cytosines have been described. However, the methods described herein could in principle use any modified nucleoside and suitable conversion procedure (i.e. single-base epigenetic conversion assay) that changes the base-pairing specificity of the modified nucleoside and thereby allows the modified base to be distinguished from the corresponding unmodified nucleoside and/or other types of modification when sequenced. For example, any conversion procedure could be used allowing any one of N⁶-methyladenine (6 mA), N⁶-hydroxymethyladenine (6hmA), or N⁶-formyladenine (6fA) to be distinguished from unmodified adenosine.

In some embodiments, the conversion procedure converts unmodified nucleosides. In some embodiments, the conversion procedure which converts unmodified nucleosides comprises bisulfite conversion. Treatment with bisulfite converts unmodified cytosine and certain modified cytosine nucleotides (e.g. 5-formyl cytosine (5fC) or 5-carboxylcytosine (5caC)) to uracil whereas other modified cytosines (e.g., 5mC and 5hmC) are not converted. Thus, where bisulfite conversion is used, the converted nucleobases are inferred as comprising one or more of unmodified cytosine, 5fC, 5caC, or other cytosine forms affected by bisulfite. The unconverted nucleobases are inferred as comprising one or more of 5mC and 5hmC. Sequencing of bisulfite-treated DNA identifies positions that are read as cytosine as being 5mC or 5hmC positions. Meanwhile, positions that are read as T are identified as being T or a bisulfite-susceptible form of C, such as unmodified cytosine, 5fC, or 5caC. Thus, performing bisulfite conversion as described herein thus facilitates identifying positions containing 5mC or 5hmC. Hence, in these embodiments, the end repair reaction can be performed with dNTPs, wherein at least one type of dNTP comprises a 5mC and/or a 5hmC, and regions synthesized during the end repair reaction can be identified as those regions comprising 5mC or a 5hmC (via C being called at these positions) at non-CpG positions. For an exemplary description of bisulfite conversion, see, e.g., Moss et al., Nat Commun. 2018; 9: 5068.

In some embodiments, the procedure which converts unmodified nucleosides comprises oxidative bisulfite (Ox-BS) conversion. This procedure first converts 5hmC to 5fC, which is bisulfite susceptible, followed by bisulfite conversion. Thus, when oxidative bisulfite conversion is used, the converted nucleobases are inferred as comprising one or more of unmodified cytosine, 5fC, 5caC, 5hmC, or other cytosine forms affected by bisulfite. The unconverted nucleobases are inferred as comprising 5mC. Sequencing of Ox-BS converted DNA identifies positions that are read as cytosine as being 5mC positions. Meanwhile, positions that are read as T are identified as being T or a bisulfite-susceptible form of C, such as unmodified cytosine, 5fC, or 5hmC. Hence, in these embodiments, the end repair reaction can be performed with dNTPs, wherein at least one type of dNTP comprises a 5mC, and regions synthesized during the end repair reaction can be identified as those regions comprising 5mC (via C being called at these positions) at non-CpG positions. Performing Ox-BS conversion thus facilitates identifying positions containing mC. For an exemplary description of oxidative bisulfite conversion, see, e.g., Booth et al., Science 2012; 336: 934-937.

In some embodiments, the procedure which converts unmodified nucleosides comprises Tet-assisted bisulfite (TAB) conversion. In TAB conversion, 5hmC is protected from conversion and 5mC is oxidized in advance of bisulfite treatment, so that positions originally occupied by 5mC are converted to U while positions originally occupied by 5hmC remain as a protected form of cytosine. For example, as described in Yu et al., Cell 2012; 149: 1368-80, β-glucosyl transferase can be used to protect 5hmC (forming 5-glucosylhydroxymethylcytosine (5ghmC)), then a TET protein such as mTet1 can be used to convert 5mC to 5caC, and then bisulfite treatment can be used to convert C and 5caC to U while 5ghmC remains unaffected. Thus, when TAB conversion is used, the converted nucleobases are inferred as comprising one or more of unmodified cytosine, 5fC, 5caC, 5mC, or other cytosine forms affected by bisulfite. The unconverted nucleobases are inferred as comprising 5hmC. Sequencing of TAB-converted DNA identifies positions that are read as cytosine as being 5hmC positions. Meanwhile, positions that are read as T are identified as being T, or a bisulfite-susceptible form of C, such as unmodified cytosine, 5mC, 5fC, or 5caC. Performing TAB conversion on a first subsample as described herein thus facilitates identifying positions containing 5hmC. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions.

In some embodiments, the conversion procedure which converts unmodified nucleosides comprises APOBEC-coupled epigenetic (ACE) conversion. In ACE conversion, an AID/APOBEC family DNA deaminase enzyme such as APOBEC3A (A3A) is used to deaminate unmodified cytosine and 5mC without deaminating 5hmC, 5fC, or 5caC. Sequencing of ACE-converted DNA identifies positions that are read as cytosine as being 5hmC, 5fC, or 5caC positions. Meanwhile, positions that are read as T are identified as being T, unmodified C, or 5mC. Performing ACE conversion as described herein thus facilitates distinguishing positions containing 5hmC from positions containing 5mC or unmodified C using the sequence reads obtained from the first subsample. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions. For an exemplary description of ACE conversion, see, e.g., Schutsky et al., Nature Biotechnology 2018; 36: 1083-1090.

Modification sensitive sequencing also includes sequencing methods which do not rely on a conversion step, wherein the base pairing specificity of a base is changed dependent on its modification status. For instance, single molecule techniques such as nanopore based sequencing and single molecule real time sequencing can be used to directly detect modified bases.

For example, some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore. One example of a nanopore sequencing system is that commercialized by Oxford Nanopore Technologies (ONT). (See e.g., (Weirather et al., F1000Research, 6:100, 2017.) ONT sequencing directly sequences a native single-stranded DNA (ssDNA) molecule by measuring characteristic current changes as the bases are threaded through the nanopore by a molecular motor protein. ONT sequencing uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “1D” reads (“template” and “complement”) by removing the adaptor. The consensus sequence of two “1D” reads is a “2D” read with a higher accuracy.

Nanopore sequencing can be used to detect base modifications including 5mC, 5hmC, 6 mA, BrdU, FdU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the modification sensitive sequencing comprises nanopore sequencing. In such embodiments, the end repair may be performed using dNTPs, which comprise 4mC, 5mC, 5hmC, 6 mA, BrdU, FdU, IdU, and/or EdU.

Another modification sensitive single molecule sequencing technique is single molecule real time sequencing (SMRT) that has been commercialized by Pacific Biosciences. SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.)

Single molecule real time sequencing can be used to detect base modifications such as 4mC, 5mC, 5hmC, 6 mA, and 8oxoG (Gouil & Keniry Essays in Biochemistry (2019) 63 639-648). Accordingly, in some embodiments, the modification sensitive sequencing comprises single molecule real time sequencing. In such embodiments, the end repair may be performed using dNTPs, which comprise 4mC, 5mC, 5hmC, 6 mA, and/or 8oxoG.

### Partitioning

In some instances, a heterogeneous nucleic acid sample is partitioned into two or more partitions (sub-samples). In some embodiments, each partition is differentially tagged. Tagged partitions can then be pooled together for collective sample prep and/or sequencing. The partitioning-tagging-pooling steps can occur more than once, with each round of partitioning occurring based on a different characteristics, and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more base modifications and without the one or more base modifications. Examples of base modifications are described elsewhere herein. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some cases, different procedures are applied to different partitions to determine different characteristics of the initial sample. The DNA of at least one partition is subjected to an end repair and modification sensitive sequencing procedure according to the methods of the disclosure described herein. In some embodiments at least one partition is not subjected to the end repair and modification sensitive sequencing procedure according to the methods of the disclosure described herein. In cases where the modification sensitive sequencing procedure comprises a conversion procedure, corresponding sequences from the converted and non-converted partitions can be compared to identify single nucleotides that have undergone conversion and therefore identify corresponding modified nucleosides in the initial sample.

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to a molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Disclosed methods herein comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

The agents used to partition populations of nucleic acids within a sample can be affinity agents, such as antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation).

In some embodiments, methylation levels can be determined using partitioning, modification-sensitive conversion such as bisulfite conversion, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner® Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be partitioned into different partitions based on the nucleic acid molecules that are bound to a specific protein or a fragment thereof and those that are not bound to that specific protein or fragment thereof.

Nucleic acid molecules can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acid molecules based on protein bound regions. Examples of methods used to partition nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with capture probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

### Amplification

Adapted DNA can be amplified (e.g. by PCR) prior to, or as part of, the modification-sensitive sequencing. For example, in modification-sensitive sequencing procedures which comprise a conversion step, the adapted DNA may be amplified after the conversion step. In modification-sensitive sequencing procedures which involve single molecule sequencing (such a nanopore-based sequencing or SMRT sequencing), there may be no amplification step.

Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids. Preferably the present methods increase the amount or number of amplified molecules relative to control methods performed with T-tailed adapters alone by at least 10, 15 or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

### Enriching, Capturing and Using Capture Probes

DNA molecules in a sample can be subject to a capture step, in which molecules having target sequences are captured for subsequent analysis. In some embodiments, methods disclosed herein comprise a step of capturing one or more sets of target regions of DNA, such as cfDNA. Capture may be performed using any suitable approach known in the art. Target capture can involve use of a bait set comprising oligonucleotide baits labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

A panel of regions targeted for enrichment can be selected such that they do not contain regions known to include the base modification used in the end repair reaction. When the end repair is performed with dNTPs comprising 5mC or 5hmC, a panel of regions targeted for enrichment may be selected such that they do not contain CpH dinucleotides which are known to be naturally methylated in the subject (e.g. humans). Such CpH dinucleotides can be identified through the use of publicly available resources (e.g. MethBank3.0: a database of DNA methylomes across a variety of species Nucleic Acids Res 2018). Such an approach has the advantage that any detected methylated CpH dinucleotides can unambiguously be attributed to regions synthesized in the end repair.

In some embodiments, capturing comprises contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing cfDNA obtained from a subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein.

In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater than the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, a captured set of DNA (e.g., cfDNA) is provided. With respect to the disclosed methods, the captured set of DNA may be provided, e.g., by performing a capturing step prior to a sequencing step as described herein. The captured set may comprise DNA corresponding to a sequence-variable target region set, an epigenetic target region set, or a combination thereof. In some embodiments, a capture step is performed prior to a conversion step or after a conversion step.

In some embodiments, a first target region set is captured (e.g., from a sample or a first subsample), comprising at least epigenetic target regions. The epigenetic target regions captured from the first subsample may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that show lower methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, a second target region set is captured from the second subsample, comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The first and second captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

In some embodiments, the DNA that is captured comprises intronic regions. In some embodiments, the intronic regions comprise one or more introns likely to differentiate DNA from neoplastic (e.g., tumor or cancer) cells and from healthy cells, e.g., non-neoplastic circulating cells. For example, an intron comprising a rearrangement known to be present in some neoplastic cells and absent from healthy cells can be used to differentiate DNA from neoplastic (e.g., tumor or cancer) cells and from healthy cells. In some embodiments, the rearrangement is a translocation.

In some embodiments, captured intronic regions have a footprint of at least 30 bp, e.g., at least 100 bp, at least 200 bp, at least 500 bp, at least 1 kb, at least 2 kb, at least 5 kb, at least 10 kb, at least 20 kb, at least 50 kb, at least 200 kb, at least 300 kb, or at least 400 kb. In some embodiments, the intronic target region set has a footprint in the range of 30 bp-1000 kb, e.g., 30 bp-100 bp, 100 bp-200 bp, 200 bp-500 bp, 500 bp-1 kb, 1 kb-2 kb, 2 kb-5 kb, 5 kb-10 kb, 10 kb-20 kb, 20 kb-50 kb, 50 kb-100 kb, 100-200 kb, 200-300 kb, 300-400 kb, 400-500 kb, 500-600 kb, 600-700 kb, 700-800 kb, 800-900 kb, and 900-1,000 kb.

Exemplary rearrangements, such as intronic translocations that can be detected using the methods described herein include but are not limited to translocations wherein at least one of the two genes involved in the translocation is a receptor tyrosine kinase. Exemplary translocation products are the BCR-ABL fusion. and fusions comprising any of ALK, FGFR2, FGFR3, NTRK1, RET, or ROS1.

In some embodiments, the DNA that is captured comprises target regions having a type-specific epigenetic variation. In some embodiments, an epigenetic target region set consists of target regions having a type-specific epigenetic variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell or tissue types but not to the extent observed in the one or more related cell or tissue types. In some embodiments, the hypermethylation variable target regions show lower methylation in healthy cfDNA than in at least one other tissue type. In some embodiments, the hypermethylation variable target regions show even higher methylation in cfDNA from a diseased cell of the one or more related cell or tissue types. In some embodiments, target regions comprise hypermethylated regions with aberrantly high copy number. In some such embodiments, the target regions are hypermethylated in healthy and diseased colon tissue and have aberrantly high copy number in pre-cancerous or cancerous colon tissue. Examples of such target regions are shown in Table 1 below.

TABLE 1
Hypermethylated target regions with aberrantly
high copy number in colon cancer or pre-cancer

| Chromosomal | Genes comprising |
|---|---|
| region with | DMRs within the |
| copy number gain | chromosomal region |
| 7p21.3-15.1 | VWDE, TWIST1, DNAH1, GPNMB, NPY, GSDME, |
| | NFE2L3, HOXA1, HOXA7, EVX1, CREB5 |
| 8q13.3-24.3 | PRDM14, MSC, TRPA1, SBSPON, CRISPLD1, |
| | ZFHX4, RALYL, MMP16, C8orf88, RUNX1T1, |
| | CDH17, GDF6, SDC2, OSR2, PABPC1, ZNF706, |
| | RIMS2, ZFPM2, SYBU, CSMD3, TRPS1, AARD, |
| | TNFRSF11B, MAL2, COL14A1, POU5F1B, |
| | ADCY8, ST3GAL1, TRAPPC9, AGO2, ADGRB1, |
| | LY6E, LY6H, GLI4, MAFA, TSTA3, |
| | PLEC, OPLAH, FOXH1 |
| 13q14.1-33.3 | ELF1, RGCC, TNFSF11, SERP2, RB1, |
| | CNMD, PCDH17, EDNRB, POU4F1, SLITRK5, |
| | GPC6, SOX21, CLDN10, STK24, ZIC2, NALCN, |
| | FGF14, MYO16, COL4A1, COL4A2, SOX1, F7 |
| 20q12-13.33 | MAFB, LPIN3, EMILIN3, PTPRT, TOX2, |
| | GDAP1L1, HNF4A, MMP9, CDH22, PREX1, |
| | ZNFX1, PARD6B, ZFP64, DOK5, FAM210B, |
| | TFAP2C, RBM38, CTCFL, GNAS, PHACTR3, |
| | CDH4, GATA5, BHLHE23, YTHDF1, NKAIN4, |
| | CHRNA4, KCNQ2, GMEB2 |

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

In some embodiments, a captured epigenetic target region set captured from a sample or subsample comprises hypomethylation variable target regions. In some embodiments, the hypomethylation variable target regions are exclusively hypomethylated in one or more related cell or tissue types. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell or tissue types but not to the extent observed in the one or more cell or tissue types. In some embodiments, the hypomethylation variable target regions show higher methylation in healthy cfDNA than in at least one other tissue type.

Without wishing to be bound by any particular theory, in an individual with cancer, proliferating or activated immune cells and/or dying cancer cells may shed more DNA into the bloodstream than immune cells in a healthy individual and/or healthy cells of the same tissue type, respectively. As such, the distribution of cell type and/or tissue of origin of cfDNA may change upon carcinogenesis. Thus, the presence and/or levels of cfDNA originating from certain cell or tissue types can be an indicator of disease. Variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set. The sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLoS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above and in WO 2020/160414.

In some embodiments, a collection of capture probes is used in methods described herein, e.g., comprising capture probes prepared by any method disclosed herein for doing so. In some embodiments, the collection of capture probes further comprises target-binding probes specific for a sequence-variable target region set and/or target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of capture probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of capture probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of capture probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the capture probes specific for the sequence-variable target region set are present at a higher concentration than the capture probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the capture probes specific for the sequence-variable target region set have a higher affinity for their targets than the capture probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39: 8740-8751 (2011); Freier et al., Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the capture probes specific for the sequence-variable target region set have modifications that increase their affinity for their targets. In some embodiments, alternatively or additionally, the capture probes specific for the epigenetic target region set have modifications that decrease their affinity for their targets. In some embodiments, the capture probes specific for the sequence-variable target region set have longer average lengths and/or higher average melting temperatures than the capture probes specific for the epigenetic target region set. These embodiments may be combined with each other and/or with differences in concentration as discussed above to achieve a desired fold difference in capture yield, such as any fold difference or range thereof described above.

In some embodiments, the capture probes comprise a capture moiety. The capture moiety may be any of the capture moieties described herein, e.g., biotin. In some embodiments, the capture probes are linked to a solid support, e.g., covalently or non-covalently such as through the interaction of a binding pair of capture moieties. In some embodiments, the solid support is a bead, such as a magnetic bead.

In some embodiments, the capture probes specific for the sequence-variable target region set and/or the capture probes specific for the epigenetic target region set are a capture probe set as discussed above, e.g., probes comprising capture moieties and sequences selected to tile across a panel of regions, such as genes.

In some embodiments, the capture probes are provided in a single composition. The single composition may be a solution (liquid or frozen). Alternatively, it may be a lyophilizate.

Alternatively, the capture probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

### Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

### CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

### Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

### Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

### Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 3. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 3. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5.

TABLE 3

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Sequencing

In general, sample nucleic acids flanked by adapters with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms. Sequencing reactions can be performed in a variety of sample processing units, which may include multiple lanes, multiple channels, multiple wells, or other mean of processing multiple sample sets substantially simultaneously. Sample processing unit can also include multiple sample chambers to enable processing of multiple runs simultaneously.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of, for example, at least 5%, 10%, 15%, 20%, 25%, 30%, 40%, 50%, 60%, 70%, or 80% of the genome. Sequence coverage can performed on, for example, at least 5, 10, 20, 70, 100, 200 or 500 different genes, or up to, for example, 5000, 2500, 1000, 500 or 100 different genes.

Simultaneous sequencing reactions may be performed using multiplex sequencing. In some cases, cell-free nucleic acids may be sequenced with at least, for example, 1000, 2000, 3000, 4000, 5000, 6000, 7000, 8000, 9000, 10000, 50000, or 100,000 sequencing reactions. In other cases cell-free nucleic acids may be sequenced with less than, for example, 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least, for example, 1000, 2000, 3000, 4000, 5000, 6000, 7000, 8000, 9000, 10000, 50000, or 100,000 sequencing reactions. In other cases, data analysis may be performed on less than, for example, 1000, 2000, 3000, 4000, 5000, 6000, 7000, 8000, 9000, 10000, 50000, or 100,000 sequencing reactions. An exemplary read depth is 1000-50000 or 1000-10000 or 1000-20000 reads per locus (base).

In general, sequencing of epigenetic target regions, e.g. to analyze a modified nucleoside profile of DNA, requires a lesser depth of sequencing than sequencing of a sequence-variable target region, e.g. for analysis of mutations. Hence, lesser sequencing depths, as described herein, may in some cases be adequate for the methods described herein.

a. Differential Depth of Sequencing

In some embodiments, nucleic acids corresponding to the sequence-variable target region set are sequenced to a greater depth of sequencing than nucleic acids corresponding to the epigenetic target region set. In some embodiments, nucleic acids corresponding to the hydroxymethylation-variable target region set are sequenced to a greater depth of sequencing than nucleic acids corresponding to at least one other target region set. For example, the depth of sequencing for nucleic acids corresponding to the sequence-variable and/or hydroxymethylation-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

In some embodiments, the captured cfDNA corresponding to the hydroxymethylation variable target region set and the captured cfDNA corresponding to the at least one other target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the hydroxymethylation variable target region set and the captured cfDNA corresponding to the at least one other target region set in the same vessel.

### Analysis

Defining Regions of the End-Repaired DNA that were Synthesized During the End Repair

In general, the methods described herein rely on the use of at least one dNTP comprising a modified base in the end repair reaction coupled with the use of modification-sensitive sequencing which can detect the modified base. This allows for regions synthesized in the end repair to be identified in the sequencing data. This is important because these synthesized regions can lead to artifactual data in typical sequencing reactions which do not control for these synthesized regions. For example, when end repair is performed with unmodified dNTPs prior to methylation-sensitive sequencing (e.g. bisulfite sequencing), the end repair can lead to 5′overhang filling, nick translation and gap filling with dCTP comprising unmodified cytosines. These unmodified cytosines may not reflect the original methylation status at these positions in the original DNA molecule (i.e. prior to the formation of the overhang, nicks and gaps) and thus the end repair can lead to artifactual methylation information. The methods disclosed herein avoid such artifactual information by identifying the sequencing data corresponding to these synthesized regions. Such regions can, e.g. then be filtered such that they are not used to classify the methylation status of the DNA molecule.

The regions synthesized in the end repair can be classified in a variety of ways and the exact approach will depend on the identity of the modified base used in the end repair reaction as well as the modification sensitive sequencing method being used. Moreover, the exact end points of the regions classified as being synthesized during end repair can be determined by the user. The basic step of the identification of the regions synthesized during the end repair reaction is the identification of the presence of the base modification in the at least one type of dNTP used in the end repair reaction.

In some embodiments, the end repair is performed with dNTP comprising 5mC or 5hmC. 5mC and 5hmC are both naturally occurring base modifications, so upon the identification of these modified bases in the sequencing data may derive from: (i) modified bases present in the original DNA molecule; or (ii) modified bases introduced in the end repair reaction. 5mC and 5hmC can, however, be classified as being introduced in the end repair reaction when they occur in a non-CpG sequence context. While CpH (i.e. CpA, CpT, CpC) methylation has been described in humans, it is thought to comprise 0.02% of total methyl-cytosine in differentiated somatic cells (Jang et al. Genes (Basel). 2017 June; 8(6): 148). As such, methylated cytosines in a CpH sequence context can confidently be attributed to regions synthesized during end repair. This is particularly the case when the disclosed methods comprise enrichment for a sequence panel wherein the panel does not comprise regions known to contain methylated CpH sites. Alternatively, the classification of whether or not a methylated CpH is part of a synthesized region can be made by accounting for: (i) the position of particular of the CpH site in a reference sequence; and/or (ii) the methylation status of the surrounding CpH sites. For example, if a CpH site is known to be methylated in nature (e.g. by comparison to reference data), then methylation detected at that CpH site can be ignored when defining regions synthesized during end repair. Similarly, detected methylation at such CpH sites can be called as the true methylation status in the DNA sample. If a CpH site known to be methylated in nature is detected as being methylated in the sequencing data, but is contained within a string of other methylated CpH sites, some of which are not known to be methylated in nature ((e.g. by comparison to reference data), the region may still be classified as being synthesized during end repair.

In embodiments wherein the end repair is performed with dNTP comprising 5mC and/or 5hmC, a region of the one or more regions of the end-repaired DNA that were synthesized during the end repair is defined as: (i) the sequence between two non-methylated cytosines which span a methylated non-CpG cytosine; and/or (ii) the sequence between a non-methylated cytosine and the end of a sequence read wherein there is no additional non-methylated cytosine between the non-methylated cytosine and the end of the sequence read. Alternatively, the one or more regions of the end-repaired DNA that were synthesized during the end repair may be defined as: (i) the sequence from a first methylated non-CpG cytosine to the last methylated non-CpG cytosines in one or more consecutive methylated non-CpG cytosines; and/or (ii) the sequence from a methylated cytosine (5mC or 5hmC) in a non-CpG context to the end of a sequence read wherein there is no non-methylated cytosine between the methylated cytosine in the non-CpG context and the end of the sequence read. The “end of the sequence read” refers to the portion of the sequence read which corresponds to the end-repaired DNA molecule and does not include, e.g. adapter sequences.

In some embodiments the end repair is performed with dNTPs comprising base modifications which are not naturally found in the subject the DNA sample derives from, or are present at only very low frequencies. For example 4mC does not occur in mammals (e.g. humans), whereas 6 mA occurs only at very low frequencies (Xiao et al. Molecular Cell Volume 71, Issue 2, 19 Jul. 2018, Pages 306-318.e7). In these embodiments, the regions of the end-repaired DNA that were synthesized during the end repair can be classified simply as any region wherein the modified base is detected. While such an approach may result in falsely classifying naturally occurring low frequency base modifications as being the result of end repair, this will be rare and may simply result in the corresponding sequence data not being used for further analysis. This is preferable to using sequence data from regions synthesized during end repair, which may contain artifactual data which may lead to false inferences regarding the corresponding DNA sample and subject.

Therefore, in some embodiments wherein the modified base is other than 5mC or 5hmC, a region of the one or more regions is defined as: (i) the sequence between two non-modified bases spanning a modified base, wherein the bases are of the same identity as the bases present in the at least one type of dNTP comprising the modified base; and/or (ii) the sequence between a non-modified base and the end of a sequence read, wherein there is no additional non-modified bases between the non-modified base and the end of the sequence read, where the non-modified bases are of the same identity as the modified base present in the at least one type of dNTP comprising the modified base. Alternatively, the one or more regions of the end-repaired DNA that were synthesized during the end repair may be defined as: (i) the sequence from a first modified to the last modified base in one or more consecutive modified bases wherein the bases are of the same identity as the bases present in the at least one type of dNTP comprising the modified base; and/or (ii) the sequence from a modified base to the end of a sequence read wherein there is no non-modified base between the modified base and the end of the sequence read where the modified base and non-modified base are of the same identity to the at least one type of dNTP comprising the modified base.

Once identified, the regions of the end-repaired DNA classified as being synthesized during the end repair may be filtered out of the sequence data such that they are not used for further analysis, such as variant calling or for determining the modification status of bases in the original DNA molecule (i.e. prior to end repair). Accordingly, in some embodiments the methods disclosed herein further comprise analyzing at least some of the sequence data corresponding to regions that are not identified as being synthesized during the end repair to detect the presence or absence of base modifications or mutations present in the DNA sample. The disclosed methods of identifying regions synthesized during end repair are advantageous over the prior art methods which use uninformed “end-clipping” because these prior art methods potentially remove regions which were not synthesized in the end repair reaction and are thus representative of the original DNA molecule.

### Exemplary Applications

The methods disclosed herein allow for detection of regions of the end-repaired DNA that were synthesized during the end repair. This information has utility in a wide range of contexts, including determining the methylation status of DNA in the DNA sample (i.e. before the end repair) and in the detection of mutations in the DNA.

The methods presented herein may be used as part of any method that benefits from obtaining an accurate modified nucleoside profile of DNA in any DNA sample and/or accurate mutation calling of DNA in any DNA sample. This is because the methods disclosed herein allow for the identification of sequencing data which corresponds to regions of an end-repaired DNA molecule that were synthesized during end repair, and thus may not be representative of the original DNA molecule. Identification of these regions avoids relying on such potentially artifactual data for subsequent analysis, such as mutation calling and/or subsequent methylation analysis.

For example, the classification of whether a variant is present or absent in a DNA molecule is dependent on whether there is double stranded support for the variant. Double stranded support refers to the presence of sequencing data derived from both DNA strands which support the presence of the variant. In synthesized regions, however, double stranded support may artificially be introduced through the end repair and/or A tailing reactions which will synthesize the region using the complementary strand as a template. This may occur when there was a mismatch in the original DNA molecule at the equivalent position, and thus the variant was not present in both strands of the original DNA molecule prior to end repair and/or A tailing. The methods disclosed herein can therefore be used to identify synthesized regions and filter variants within those regions which would otherwise erroneously be classified as having double stranded support. The use of the disclosed methods in variant calling is particularly advantageous when the modification sensitive sequencing method used does not require the conversion of unmethylated cytosines and are thus compatible with high sensitivity variant calling. Accordingly, in some embodiments, the methods disclosed herein are used for detecting SNVs, wherein the modification sensitive sequencing is nanopore-based sequencing, single-molecule real time (SMRT) sequencing or Tet-assisted pyridine borane sequencing (TAPS).

One important exemplary application of the methods of the disclosure is using the resulting sequencing data in diagnosing and prognosing cancer or other genetic diseases or conditions.

Hence, in some embodiments, methods described herein comprise identifying or predicting the presence or absence of DNA produced by a tumor (or neoplastic cells, or cancer cells), determining the probability that a test subject has a tumor or cancer, and/or characterizing a tumor, neoplastic cells or cancer as described herein.

The present methods can be used to diagnose presence or absence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. In some embodiments, target regions are analyzed to determine whether they show methylation characteristics of tumor cells or cells that do not ordinarily contribute significantly to cfDNA and/or target regions are analyzed to determine whether they show methylation characteristic of tumor cells or cells that do not ordinarily contribute significantly to cfDNA.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Additionally, if a cancer is observed to be in remission after treatment, the present methods can be used to monitor residual disease or recurrence of disease.

Typically, the disease under consideration is a type of cancer, such as any referred to herein. The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, such as 5mC and 5mC profiles. Hence, the present methods can in some cases be used in combination with methods used to detect other genetic/epigenetic variations, e.g. in a method of detecting or characterizing a cancer or other methods described herein.

In some embodiments, a method described herein comprises identifying the presence of target regions and/or DNA produced by a tumor (or neoplastic cells, or cancer cells) or by precancer cells. In some embodiments, a method described herein comprises determining the level of target regions and/or identifying the presence of DNA produced by a tumor (or neoplastic cells, or cancer cells) or by precancer cells. In some embodiments, determining the level of target regions comprises determining either an increased level or decreased level of target regions, wherein the increased or decreased level of target regions is determined by comparing the level of target regions with a threshold level/value.

Genetic and/or epigenetic data can also be used for characterizing a specific form of cancer. Cancers are often heterogeneous in both composition and staging. Genetic and/or epigenetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic and/or epigenetic profile of cfDNA derived from the subject, wherein the genetic and/or epigenetic profile comprises a plurality of data resulting from copy number variation and rare mutation analyses. In some embodiments, an abnormal condition is cancer, e.g. as described herein. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer or precancer, or has a metastasis, that is related to changes in proportions of types of immune cells.

The present methods can be used to generate or profile, fingerprint or set of data that is a summation of genetic and/or epigenetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, the methods can provide a measure of the extent of DNA damage through the quantification of the regions synthesized during the end repair, the methods disclosed herein can also be used to quantify the level of DNA damage present in the original DNA sample. This is because the level of end repair will depend in part on the amount of DNA damage (e.g. gaps, nicks and overhangs) present in the DNA because it is this damage which can act as the priming sites for synthesis in the end repair (see FIGS. 1-6 and the corresponding descriptions).

In some embodiments, the method further comprises calculating a synthesis index which is a quantitative measure of the regions synthesized in the end repair. The synthesis index may be on a molecule level and/or a sample level. The synthesis index may be the proportion of sequencing data which corresponds to synthesized regions. In some embodiments, the method further comprises comparing the synthesis index to one or more reference values to classify the DNA sample. The classification may be whether the DNA sample derives from a subject with or without cancer. The reference values may be derived from one or more control DNA samples which are known to have a specific properties, such as being derived from a subject known to have cancer, e.g. a specific type of cancer. The reference values may be obtained by performing the method used to obtain the synthesis index on control samples (i.e. using the same end repair, ligation and sequencing methods).

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a subject and/or classifying a subject as being a candidate for a subsequent cancer treatment.

Methods of Determining a Risk of Cancer Recurrence in a Subject and/or Classifying a Subject as being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting a sample (such as DNA, such as DNA originating or derived from a tumor cell) from the subject diagnosed with the cancer at one or more preselected timepoints following one or more previous cancer treatments to the subject. The subject may be any of the subjects described herein. The sample may comprise chromatin, cfDNA, or other cell materials. The sample, such as the DNA sample, may be a tissue sample.

Any of such methods may comprise capturing a plurality of sets of target regions from DNA from the subject, wherein the plurality of target region sets comprises a sequence-variable target region set and an epigenetic target region set, whereby a captured set of DNA molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as genomic regions of interest and target regions, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, sequence-variable target region sequences are obtained, and determining the cancer recurrence score may comprise determining at least a first subscore indicative of the amount of SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, epigenetic target region sequences are obtained, and determining the cancer recurrence score comprises determining a second subscore indicative of the amount of molecules (obtained from the epigenetic target region sequences) that represent an epigenetic state different from DNA found in a corresponding sample from a healthy subject (e.g., cfDNA found in a blood sample from a healthy subject, or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer (such as with a metastasis), e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or more of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a subscore indicative of the amount of SNVs, insertions/deletions, CNVs and/or fusions present in sequence-variable target region sequences and a subscore indicative of the amount of abnormal molecules in epigenetic target region sequences, and combining the subscores to provide the cancer recurrence score. Where the subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy.

As disclosed herein, in some embodiments, quantities of each of a plurality of cell types, such as immune cell types, are determined based on sequencing and analysis (such as determination of epigenetic and/or genomic signatures) of DNA isolated from at least one sample comprising cells (such as a tissue sample or a blood sample, e.g., a whole blood sample, a buffy coat sample, a leukapheresis sample, or a PBMC sample) from a subject. In some embodiments, differences in levels and/or presence of particular genetic and/or epigenetic signatures in DNA isolated from blood samples from a subject can be used to quantify cell types, such as immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples (such as a tissue sample or a blood sample, e.g., a whole blood sample, a buffy coat sample, a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a tissue sample or a blood sample, e.g., a whole blood sample, a buffy coat sample, a leukapheresis sample, or a PBMC sample) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one tissue sample and/or at least one blood sample, e.g., a whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### Therapies and Related Administration

In certain embodiments, the methods disclosed herein relate to identifying and administering customized therapies, such as customized therapies to patients. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In some embodiments, therapy is customized based on the status of a nucleic acid variant as being of somatic or germline origin. In some embodiments, determination of the levels of particular cell types, e.g., immune cell types, including rare immune cell types, facilitates selection of appropriate treatment.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject. In some embodiments, determination of the metastasis site facilitates selection of appropriate treatment.

In some embodiments, therapy is customized based on the status of a detected nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

The disclosed methods can include evaluating (such as quantifying) and/or interpreting at least one cell material released from a potential metastasis site (such as at least one cell material in a sample from a subject) and/or cell types that contribute to DNA, such as cfDNA, in one or more samples collected from a subject at one or more timepoints in comparison to a selected baseline value or reference standard (or a selected set of baseline values or reference standards). A baseline value or reference standard may be a presence or level of at least one cell material and/or a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a presence or level of at least one cell material and/or a quantity of cell types measured with respect to one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, methods are provided for monitoring a response (such as a change in disease state, such as a presence or absence of a metastasis in a subject, such as measured by assessing a presence or level of at least one cell material released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples are collected from a subject at least once per week, such as on 1-4 days, 1-2 days, or on 1, 2, 3, 4, 5, 6, or 7 days per week. In certain embodiments, one or more samples are collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### Kits

Also provided are kits for use in the methods as described herein. In some embodiments, a kit comprises a first reagent for end repair to generate end-repaired DNA, wherein the first reagent comprises at least one type of dNTP that comprises a modified base. In some embodiments, the kit further comprises a second reagent for ligating adapters to the end-repaired DNA to generate adapted DNA, wherein the second reagent also seals nicks present in the end-repaired DNA. In some embodiments, the kit further comprises a third reagent for modification-sensitive sequencing that is capable of identifying the base modification in the at least one type of dNTP. The kit may comprise the first, second, and/or third reagents and additional elements as discussed below and/or elsewhere herein. In some embodiments, a kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adaptors having distinct molecular barcodes and identical sample barcodes. The library adaptors may not be sequencing adaptors. For example, the library adaptors do not include flow cell sequences or sequences that permit the formation of hairpin loops for sequencing. The different variations and combinations of molecular barcodes and sample barcodes are described throughout, and are applicable to the kit. Further, in some cases, the adaptors are not sequencing adaptors. Additionally, the adaptors provided with the kit can also comprise sequencing adaptors. A sequencing adaptor can comprise a sequence hybridizing to one or more sequencing primers. A sequencing adaptor can further comprise a sequence hybridizing to a solid support, e.g., a flow cell sequence. For example, a sequencing adaptor can be a flow cell adaptor. The sequencing adaptors can be attached to one or both ends of a polynucleotide fragment. In some cases, the kit can comprise at least 8 different library adaptors having distinct molecular barcodes and identical sample barcodes. The library adaptors may not be sequencing adaptors. The kit can further include a sequencing adaptor having a first sequence that selectively hybridizes to the library adaptors and a second sequence that selectively hybridizes to a flow cell sequence. In another example, a sequencing adaptor can be hairpin shaped. For example, the hairpin shaped adaptor can comprise a complementary double stranded portion and a loop portion, where the double stranded portion can be attached (e.g., ligated) to a double-stranded polynucleotide. Hairpin shaped sequencing adaptors can be attached to both ends of a polynucleotide fragment to generate a circular molecule, which can be sequenced multiple times. A sequencing adaptor can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adaptor can comprise 20-30, 20-40, 30-50, 30-60, 40-60, 40-70, 50-60, 50-70, bases from end to end. In a particular example, the sequencing adaptor can comprise 20-30 bases from end to end. In another example, the sequencing adaptor can comprise 50-60 bases from end to end. A sequencing adaptor can comprise one or more barcodes. For example, a sequencing adaptor can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adaptors can be blunt ended and Y-shaped and can be less than or equal to 40 nucleic acid bases in length. Other variations of the can be found throughout and are applicable to the kit.

### Computer Systems

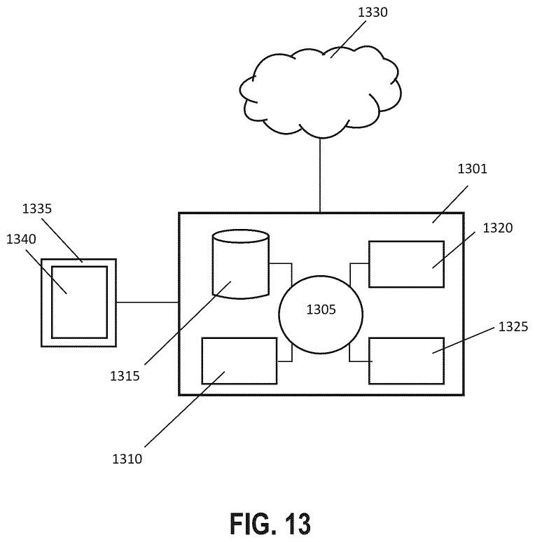

Methods of the present disclosure can be implemented using, or with the aid of, computer systems. FIG. 13 shows a computer system 1301 that is programmed or otherwise configured to implement the methods of the present disclosure. The computer system 1301 can regulate various aspects sample preparation, sequencing, and/or analysis. In some examples, the computer system 1301 is configured to perform sample preparation and sample analysis, including (where applicable) nucleic acid sequencing, e.g., according to any of the methods disclosed herein.

The computer system 1301 includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage, and/or electronic display adapters. The memory 1310, storage unit 1315, interface 1320, and peripheral devices 1325 are in communication with the CPU 1305 through a communication network or bus (solid lines), such as a motherboard. The storage unit 1315 can be a data storage unit (or data repository) for storing data. The computer system 1301 can be operatively coupled to a computer network 1330 with the aid of the communication interface 1320. The computer network 1330 can be the Internet, an internet and/or extranet, or an intranet and/or extranet that is in communication with the Internet. The computer network 1330 in some cases is a telecommunication and/or data network. The computer network 1330 can include one or more computer servers, which can enable distributed computing, such as cloud computing. The computer network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. Examples of operations performed by the CPU 1305 can include fetch, decode, execute, and writeback.

The storage unit 1315 can store files, such as drivers, libraries, and saved programs. The storage unit 1315 can store programs generated by users and recorded sessions, as well as output(s) associated with the programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet. Data may be transferred from one location to another using, for example, a communication network or physical data transfer (e.g., using a hard drive, thumb drive, or other data storage mechanism).

The computer system 1301 can communicate with one or more remote computer systems through the network 1330. For embodiment, the computer system 1301 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1301 via the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine-readable code can be provided in the form of software. During use, the code can be executed by the processor 1305. In some cases, the code can be retrieved from the storage unit 1315 and stored on the memory 1310 for ready access by the processor 205. In some situations, the electronic storage unit 1315 can be precluded, and machine-executable instructions are stored on memory 1310.

In an aspect, the present disclosure provides a non-transitory computer-readable medium comprising computer-executable instructions which, when executed by at least one electronic processor, perform at least a portion of a method described herein. For example, the method may comprise: (a) ligating the DNA to oligonucleotide adapters, wherein the adapters comprise quality control nucleosides that include modified nucleosides, wherein the quality control nucleosides have the same nucleoside identity and the same or a different modification status to modified nucleosides to be detected in the DNA, and wherein the modification status of the quality control nucleosides is known; (b) subjecting the adapted DNA, or a subsample thereof, to a conversion procedure that changes the base pairing specificity of the quality control nucleosides or does not change the base pairing specificity of the quality control nucleosides, depending on the modification status of the nucleosides, wherein the conversion procedure comprises enzymatic protection of unmodified cytosines in the DNA followed by deamination of unprotected modified cytosines, and wherein the conversion procedure is selected to (i) change the base pairing specificity of adapted DNA nucleosides having the same nucleoside identity and modification status as quality control nucleosides in the adapters, and not change the base pairing specificity of adapted DNA nucleosides having the same nucleoside identity as quality control nucleosides in the adapters but a different modification status; and/or (ii) not change the base pairing specificity of adapted DNA nucleosides having the same nucleoside identity and modification status as quality control nucleosides in the adapters, and change the base pairing specificity of adapted DNA nucleosides having the same pairing identity as quality control nucleosides in the adapters but a different modification status; (c) sequencing the adapted DNA after conversion step (b); (d) using the sequence data obtained in step (c) to determine base pairing specificity conversion of the quality control nucleosides in the adapters; and (e) using the base pairing specificity conversion of the quality control nucleosides in the adapters as a quality control measure for conversion step (b), wherein sub-optimal conversion of adapter quality control nucleosides following a conversion procedure of step (b)(i) and/or erroneous conversion of adapter quality control nucleosides following a conversion procedure of step (b)(ii) predicts false negative and/or false positive detection of modified nucleosides in the DNA sample.

The code can be pre-compiled and configured for use with a machine have a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

The following examples are provided to illustrate certain aspects of the disclosed methods. The examples do not limit the disclosure.

### Example 1—End-Repair Results in the Synthesis of Regions of the Original DNA Molecule

Human DNA was subjected to a modification sensitive sequencing method sensitive for cytosine methylation. In order to evidence the synthesis of regions of a DNA molecule during end repair and A-tailing reactions, sequence reads predicted to come from the same original DNA molecule were identified. The library preparation workflow maintained directionality such that reads from the forward strand (“forward pair”) and reads from the reverse strand (“reverse pair”) could be identified.

Genomic regions that were known to be fully methylated in humans were analysed (positive control regions). The use of these positive control regions meant that any detected unmethylated cytosines in these regions could be attributed to regions synthesized during end repair. From these data, the extent of end repair on each individual strand was estimated as the number of consecutive unmethylated bases into strand from 3′end. This analysis was performed from only the 3′end as it is the 3′end which is extended in the end repair reaction to fill in 5′overhangs. End repair would not be expected to result in the synthesis of regions at the 5′ends of DNA molecules because at 3′ overhangs the 3′ends are resected to form a blunt end.

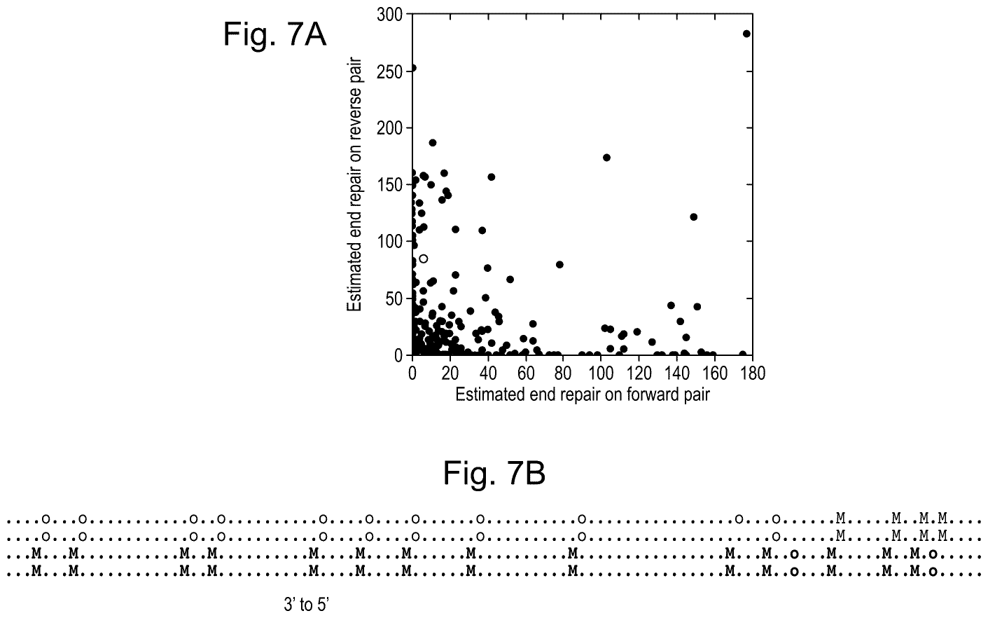

The plot shown in FIG. 7A shows the estimated end repair in both the forward pair and the reverse pair. The plot shows a significant and asymmetric methylation loss from 3′end of forward and reverse strands of positive control regions.

The sequence diagram in FIG. 7B shows the exemplary aligned forward (bottom two sequences) and reverse strands (top two sequences) of a positive control region pair. Aligned reads are shown, with ‘.’ indicating any non-cytosine base, ‘o’ and ‘M’ indicating unmethylated and methylated cytosines, respectively. These sequences show a significant methylation loss observed on the reverse (top) strands, which indicates nick/gap induced end-repair which extends over a significant portion of the molecule.

These data demonstrate that a significant portion of the original DNA molecules were synthesized during the end repair reaction, which leads to methylation data which is not representative of the original DNA molecule. Moreover, if these sequence data were used to in variant calling analysis, it could result in falsely classifying variants within the synthesized regions having double stranded support in the original DNA molecule.

### Example 2—End Repair/A-Tailing with 5mCTP Evidences Internal Fill-In

A single tube end-repair/A-tailing reaction was performed in the presence of dCTP comprising 5mC (5mCTP) in place of dCTP comprising unmodified C (dCTP). Accordingly, all dCTPs incorporated during the end repair and A-tailing would comprise 5mC. A control reaction was also performed in the absence of modified bases. Adapters were then ligated to the end-repaired and A-tailed DNA, which was then subjected to methylation-sensitive sequencing.

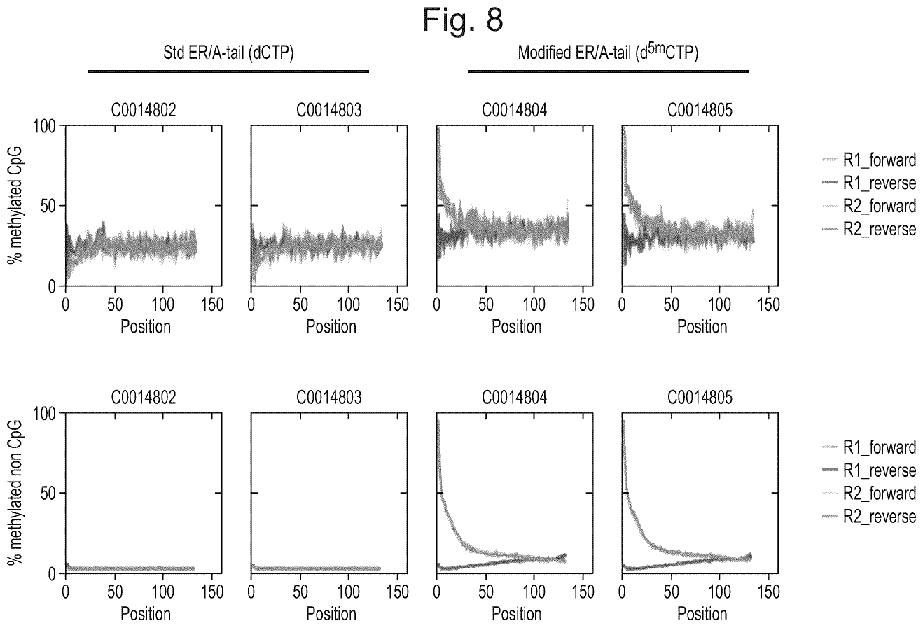

FIG. 8 shows M-bias plots of the sequencing data from the samples where the end-repair/A-tailing was performed with dCTP and with 5mCTP. These show the percentage of methylated cytosines detected in a CpG context (top row) throughout a sequence read, and the percentage of methylated cytosines detected in a non-CpG context (bottom row). As expected, the level of methylation at non-CpG positions is almost zero throughout the forward and reverse reads when the end repair/A-tailing is performed with dCTP, reflecting the low frequency of non-CpG methylation in nature.

In contrast, when the end-repair/A-tailing with 5mCTP, the level of methylated non-CpGs detected is significant, which reflects regions synthesised during the end-repair/A-tailing. The measured methylation at both CpG and non-CpG positions is high at the beginning of the “read 2” indicating significant 3′fill-in. The data also show that there is also significant synthesis of internal regions (e.g. through nick translation), which can be seen by looking at the non-CpG methylation which, along molecule is ˜0% when end-repair/A-tailing is performed with dCTP, but increases to ˜10-20% with 5mCTP used in the end-repair/A-tailing reaction.

### Example 3—Blunt End Ligation Reduces Nick Translation Compared to Single Tube End Repair a Tailing

In order to determine the effects of single tube end repair/A-tailing on the synthesis of regions of the DNA molecules, blunt ended ligation was used as a comparator. Blunt end ligation was performed by blunting the DNA with T4 DNA polymerase using dCTP in the end repair reaction, followed by ligation of blunt adapters without T overhangs. These adapted DNA were sequenced using methylation sensitive sequencing, and the resulting data was compared to equivalent experiments using single tube end repair/A-tailing with dCTP and sticky-end ligation.

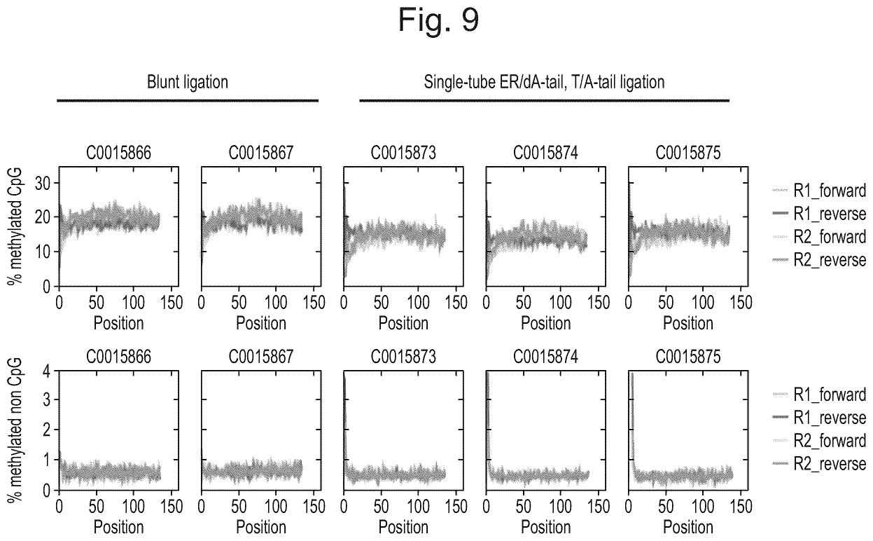

FIG. 9 shows the M-bias plots of the sequencing data from these samples. These show the percentage of methylated cytosines detected in a CpG context (top row) throughout a sequence read, and the percentage of methylated cytosines detected in a non-CpG context (bottom row). The CpG methylation level is elevated through the internal portion of the molecule with blunt end ligation versus single tube end repair/A-tailing with dCTP and sticky-end ligation, ˜20% vs ˜15% methylated CpG. This indicates that there is a lower level of synthesized regions when using blunt end ligation, and thus a lower level of methylation loss in the sequencing data. In non-CpG the methylation level is consistent between blunt ligation and sticky-end ligation as expected, because non-CpG methylation is rare.

Next, the same blunt ended and sticky-end ligation reactions were performed using 5mCTP in the end repair reaction, which results in incorporation of 5mCTP in synthesized regions in both CpG and non-CpG context.

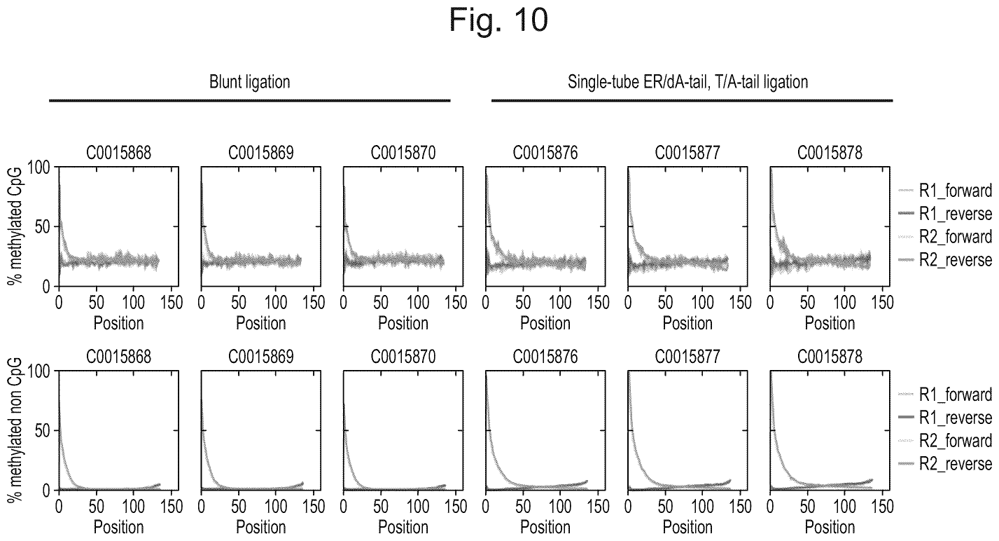

FIG. 10 shows the M-bias plots of the sequencing data from these samples. These show the percentage of methylated cytosines detected in a CpG context (top row) throughout a sequence read, and the percentage of methylated cytosines detected in a non-CpG context (bottom row). These data show that conducting end-repair with 5mCTP can be used to identify end-repair synthesized regions, which may contain artifactual data, in blunt end ligation and single-tube ER/dA-tail library preparation process. The plots show a detectable increase in methylated CpGs and non-CpGs at the start of read2, which identifies 3′end fill at 5′overhangs during the end-repair reaction.

The plots also show that blunt end ligation results in a reduction of internal synthesized regions through the reduced increase in non-CpG methylation throughout the sequence read, when compared to the single-tube ER/dA-tail library preparation process. This is thought to be due to a reduction in nick translation in the workflow using blunt end ligation compared to the single-tube ER/dA-tail library preparation process.

Together, these data show that end repair and A-tailing can lead to the synthesis of DNA regions, and that these regions may contain artifactual data which is not representative of the original DNA molecule. These data also show that dNTPs comprising a modified base can be used to identify such regions, which in turn enables such artifactual data to be controlled for.

### Example 4—mCTP is Efficiently Incorporated into DNA Molecules

In order to determine the effects of incorporation of mCTP on the efficiency of synthesis of regions of the DNA molecules, CTP was used as a comparator. End-repair/A-tailing reactions were performed in the presence of dCTP comprising 5mC (mCTP) in place of unmodified dCTP. Accordingly, all dCTPs incorporated during the end repair and A-tailing comprise 5mC. A control reaction was also performed in the absence of modified bases.

The end-repaired and A-tailed DNA was then subjected to methylation-sensitive sequencing. Comparisons of methylation rates in positive and negative control regions as well as specificity were evaluated. As a proxy for sensitivity, mCpG rates in 52 genomic regions known to harbor methylated CpGs were measured. As a proxy for specificity, mCpG rates in 148 genomic regions known to lack CpG methylation were measured. For samples without mCTP, sequences were trimmed by a fixed length of 40 bp, meaning that sequence within 40 bp of an adapter junction was excluded from analysis of methylation. For samples with mCTP, mCTP-aware trimming was performed. The trimming algorithm was used to remove the part of the molecule that has methylated non-CpGs (i.e., methylated Cs that are not followed by a G). According to the algorithm, molecules containing a plurality of methylated non-CpGs were flagged as needing trimming. The cut point for trimming was determined as the position that provides a highest quantitative measure of unmethylated non-CpGs in the retained region in relation to methylated non-CpGs in the trimmed region. As a result of the trimming, the read was truncated to only use positions 5′ of the cut point in downstream analyses. Note that the trimming happened respecting 5′->3′ directionality of the molecule as sequenced, regardless of orientation in the genome. In other words, “+” strand reads were trimmed at the genomic 3′ end, and “−” strand reads were trimmed at the genomic 5′ end, but both of these were the 3′ end of the original molecule.

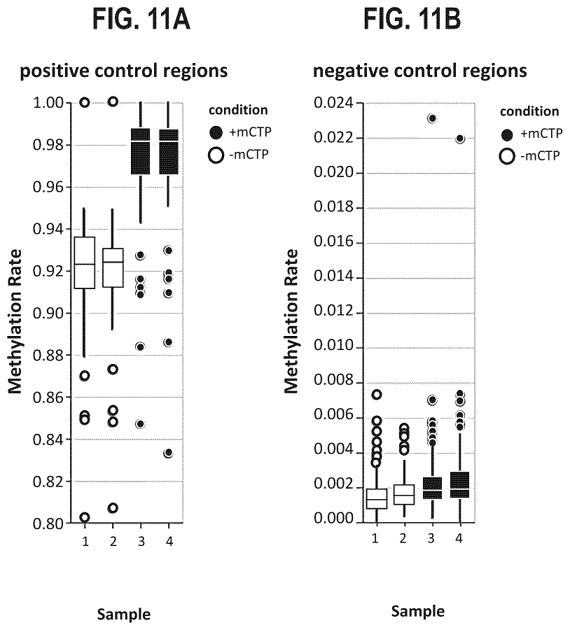

FIG. 11A shows that positive control region methylation was ˜6% higher with mCTP compared to CTP (98% vs. 92%). FIG. 11B shows negative control region methylation was also slightly higher with mCTP but the false positivity rate was ˜1-2 per 1000 in both conditions.

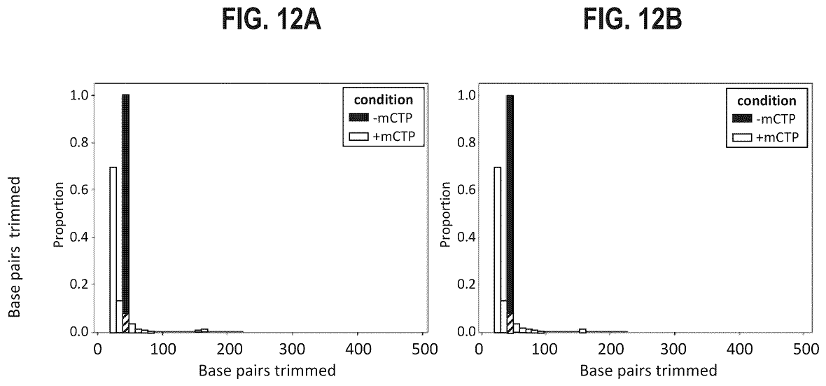

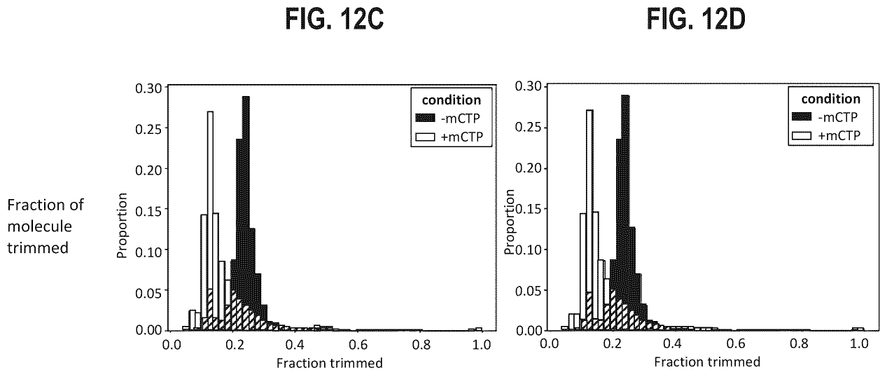

FIGS. 12A-12D show that the mCTP end trimming resulted in less trimming for most molecules compared to the fixed 40 bp trimming. Therefore, the improvement in positive control region methylation with mCTP was from the removal of end repair artifacts from the small population of molecules with >40 bp trimmed and was not that the 40 bp trim was overall too little trimming. An average of ˜32 bp was trimmed with mCTP trimming compared to 40 bp without mCTP also resulted in maintaining more bases for methylation analysis, and thereby improved molecular recovery. With mCTP-aware trimming, 15% of molecules had >40 bp trimmed meaning that a fixed trim of 40 bp was under trimming 15% of molecules. mCTP-end repair improved molecular recovery and maintained more bases for methylation analysis.

### Example 5—Analysis of cfDNA to Detect the Presence Absence of Cancer

A cfDNA sample is analyzed as described above. Briefly, the cfDNA sample is subjected to end repair to generate end-repaired DNA performed with dNTPs, where at least one type of dNTP comprises a modified base. Regions of DNA that were synthesized during the end repair comprising regions with a modified base are identified. The identified regions may be filtered from those regions which would otherwise erroneously be classified as having double stranded support. Identified regions are analyzed and combined with analysis of sequence-variable target region sequences, which are analyzed by detecting genomic alterations such as SNVs, insertions, deletions and fusions that can be called with enough support that differentiates real tumor variants from technical errors (for e.g., end repair errors) to produce a final tumor present/absent call.

1. A method comprising:
(a) subjecting a DNA sample to end repair to generate end-repaired DNA, wherein the end repair is performed with deoxynucleotide triphosphates (dNTPs), wherein at least one type of dNTP comprises a modified base;
(b) performing a ligation reaction to ligate adapters to the end-repaired DNA to generate adapted DNA, wherein the ligation reaction also seals nicks present in the end-repaired DNA;
(c) subjecting the adapted DNA to modification-sensitive sequencing to obtain sequencing data derived from the DNA sample, wherein the modification-sensitive sequencing is capable of identifying the base modification in the at least one type of dNTP; and
(d) analyzing the sequence data obtained in step (c) to identify one or more regions of the end-repaired DNA that were synthesized during the end repair by the presence of the base modification in the at least one type of dNTP used in step (a).

2. The method of claim 1, (i) wherein the end repair is performed with a DNA polymerase which does not have 5′-3′ exonuclease activity and/or is not a strand displacing DNA polymerase, optionally wherein the DNA polymerase is T4 DNA polymerase, T7 DNA polymerase or Klenow fragment, or (ii) wherein the end repair is performed with a DNA polymerase which has 5′-3′ exonuclease activity and/or is a strand displacing DNA polymerase.

3. (canceled)

4. (canceled)

5. The method of claim 1, wherein the at least one type of dNTP which comprises a modified base modified base includes a dNTP comprising 4-methylcytosine (4mC), a dNTP comprising 5-methylcytosine (5mC), a dNTP comprising 5-hydroxymethyl-cytosine (5hmC), a dNTP comprising N6-methyladenosine (6 mA), a dNTP comprising bromodeoxyuridine (BrdU) and/or a dNTP comprising 8-oxoguanine (8oxoG).

6. The method of claim 1, further comprising between steps (a) and (b) performing an A-tailing reaction.

7. The method of claim 6, wherein:
(i) the end-repair and the A-tailing reaction are performed in the same reaction mixture, optionally wherein the end-repair and the A-tailing reaction are performed a single tube and/or optionally wherein the end-repair and the A-tailing reaction are performed without an intervening clean-up step; or
(ii) the end-repair and the A-tailing reaction are performed as separate reactions, wherein a reaction clean-up step is performed after the end-repair and before the A-tailing reaction, optionally wherein the reaction clean-up step removes unincorporated dNTPs.

8. The method of claim 7, wherein:
(i) the A-tailing is performed using a DNA polymerase that does not possess 5′-3′ exonuclease activity and/or is not a strand displacing DNA polymerase, optionally wherein the DNA polymerase is HemoKlen Taq;
(ii) the A-tailing is performed using a thermostable DNA polymerase; or
(iii) the A-tailing is performed using Tag DNA polymerase, Tfl DNA Polymerase, Bst DNA Polymerase, Large Fragment or Tth DNA polymerase.

9. (canceled)

10. (canceled)

11. (canceled)

12. (canceled)

13. The method of claim 7, wherein:
(i) the A-tailing is performed using a DNA polymerase that does not possess 3′-5′ exonuclease activity, optionally wherein the DNA polymerase is Klenow Fragment lacking 3′-5′ exonuclease activity;
(ii) the A-tailing is performed using a DNA polymerase that possesses 5′-3′ exonuclease activity and/or is a strand displacing DNA polymerase: or
(iii) the A tailing reaction is performed at a higher temperature than the end repair, optionally wherein the end repair is performed at about 15-35° C. and/or the A tailing is performed at a temperature over about 60° C., further optionally wherein the temperature over 60° C. is about 60° C.-75° C.

14. (canceled)

15. (canceled)

16. The method of claim 1, wherein the ligation reaction is a blunt-end ligation reaction or between steps (a) and (b) performing an A-tailing reaction and wherein the ligation reaction is a sticky-end ligation reaction.

17. (canceled)

18. The method of claim 1, wherein the modification-sensitive sequencing comprises a conversion procedure that changes the base pairing specificity of the base or does not change the base pairing specificity of the base, depending on the modification status of the base.

19. The method of claim 18, (i) wherein the modified base is a methylated cytosine and wherein the conversion procedure converts methylated cytosines, wherein the conversion procedure is Tet-assisted conversion with a substituted borane reducing agent, optionally wherein the substituted borane reducing agent is 2-picoline borane, borane pyridine, tert-butylamine borane, or ammonia borane or (ii) wherein the modified base is a methylated cytosine and wherein the conversion procedure converts unmethylated cytosines, wherein the conversion procedure is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq) or single-enzyme 5-methylctyosine sequencing (SEM-seq) method.

20. (canceled)

21. The method of claim 1, wherein the modification-sensitive sequencing comprises (i) nanopore-based sequencing, optionally wherein the at least one type of dNTP which comprises a modified base includes a dNTP comprising 4mC, a dNTP comprising 5mC, a dNTP comprising 5hmC, a dNTP comprising 6 mA, a dNTP comprising BrdU, dUTP, a dNTP comprising FldU, a dNTP comprising IdU, and/or a dNTP comprising EdU or (ii) single-molecule real time (SMRT) sequencing, optionally wherein the at least one type of dNTP which comprises a modified base includes a dNTP comprising a 4mC, a dNTP comprising 5mC, a dNTP comprising 5hmC, a dNTP comprising 6 mA, and/or a dNTP comprising 8oxoG.

22. (canceled)

23. (canceled)

24. The method of claim 1, wherein the modified base is 5mC or 5hmC.

25. (canceled)

26. The method of claim 1, wherein analyzing the sequence data obtained in step (c) comprises the identification of regions comprising the modified base in non-CpG sequence contexts and classifying these regions as regions that were synthesized during the end repair or analyzing at least some of the sequence data corresponding to regions that are not identified as being synthesized during the end repair to detect the presence or absence of base modifications or mutations present in the DNA sample.

27. The method of claim 1, wherein the modified base is other than 5mC or 5hmC, and a region of the one or more regions is defined as:
(i) the sequence between two non-modified bases spanning a modified base, wherein the bases are of the same identity to the modified bases present in the at least one type of dNTP; and/or
(ii) the sequence between a non-modified base and the end of a sequence read, wherein there is no additional non-modified bases between the non-modified base and the end of the sequence read, where the non-modified bases are the same identity as the modified base present in the at least one type of dNTP; or
wherein the modified base is a methylated cytosine, such as 5mC or 5hmC, a region of the one or more regions is defined as:
(i) the sequence between two non-methylated cytosines which span a methylated non-CpG cytosine; and/or
(ii) the sequence between a non-methylated cytosine and the end of a sequence read wherein there is no additional non-methylated cytosine between the non-methylated cytosine and the end of the sequence read.

28. (canceled)

29. The method of claim 1, further comprising: (i) filtering out sequence data from the one or more regions identified as being synthesized during the end repair such that these sequence data are not used for subsequent analysis; or (ii) flagging sequence data from the one or more regions identified as being synthesized during the end repair as potentially containing artifactual sequence data that may not be representative of the DNA sample.

30. (canceled)

31. The method of claim 1, wherein:
(i) the method is for detecting the methylation status of cytosines in the DNA sample, and wherein the analyzing the sequence data comprises filtering out the one or more regions of the end-repaired DNA that are identified as being synthesized during the end repair such that the one or more regions are not used to determine the methylation status of cytosines in the DNA sample; or
(ii) the method is for detecting the single nucleotide variants (SNVs) in the DNA sample, and wherein the analyzing the sequence data comprises classifying all base calls within the one or more regions as not having double stranded support.

32. (canceled)

33. The method of claim 1, wherein the method further comprises quantifying the DNA damage in the DNA sample through the identification of the one or more regions of the end-repaired DNA that were synthesized during the end repair, optionally wherein the level of DNA damage is used to predict whether or not a portion of the DNA in the DNA sample is derived from cancerous cells.

34. (canceled)

35. The method of claim 1, wherein the DNA sample comprises cell-free DNA (cfDNA) and the method further comprises analyzing the sequence data obtained in step (c) to determine a level of measured artifacts in the cfDNA, and optionally (i) predicting whether or not a portion of the cfDNA is derived from cancerous cells using the level of measured artifacts and/or the comparison of the level of measured artifacts to one or more reference levels or (ii) determining a likelihood that a portion of the cfDNA is derived from cancerous cells using the level of measured artifacts and/or the comparison of the level of measured artifacts to one or more reference levels.

36. (canceled)

37. (canceled)

38. The method of claim 1, wherein the DNA sample comprises cell-free DNA or DNA from formalin fixed paraffin embedded samples.

39.-48. (canceled)

49. A kit comprising:
a) a first reagent for end repair to generate end-repaired DNA, wherein the first reagent comprises at least one type of dNTP that comprises a modified base;
b) a second reagent for ligating adapters to the end-repaired DNA to generate adapted DNA, wherein the second reagent also seals nicks present in the end-repaired DNA;
c) a reagent for modification-sensitive sequencing that is capable of identifying the base modification in the at least one type of dNTP and/or a DNA polymerase for incorporating the first reagent into DNA during end repair; and
d) library adaptors having distinct molecular barcodes.

50.-63. (canceled)
